(19) United States
(12) Patent Application Publication
(10) Pub. No.: US 2025/0339642 A1
(43) Pub. Date: Nov. 6, 2025

(54) CATHETERS

(71) Applicant: ConvaTec Limited, Flintshire (GB)

(72) Inventors: David Pollard, Merseyside (GB); Rachel Pytel, Newton, MA (US); Gavin Roberts, Wiltshire (GB)

(21) Appl. No.: 19/265,223

(22) Filed: Jul. 10, 2025

Related U.S. Application Data
(63) Continuation of application No. PCT/GB2024/050068, filed on Jan. 11, 2024.
(60) Provisional application No. 63/438,620, filed on Jan. 12, 2023.
(60) Provisional application No. 63/503,769, filed on May 23, 2023.

(30) Foreign Application Priority Data
Feb. 10, 2023 (GB) 2301916.9
Jul. 14, 2023 (GB) 2310824.4

(51) Int. Cl. A61M 25/00; A01N 59/00; A01P 1/00; A61L 2/18; A61L 29/04; A61L 101/06

(52) U.S. Cl. CPC A61M 25/0017; A01N 59/00; A01P 1/00; A61L 2/18; A61L 29/041; A61L 2101/06; A61L 2202/122; A61L 2202/24; A61M 2025/0019

(57) ABSTRACT

A reusable urinary catheter kit that has a container that has a urinary catheter and a medium. The reusable urinary catheter kit also has at least one chlorine-containing species. The container of the reusable urinary catheter kit is resealable. Further, the catheter in the reusable urinary catheter kit is an intermittent urinary catheter.

### TECHNICAL FIELD OF THE INVENTION

The present invention relates to reusable urinary catheters and to sterilisation/lubrication of reusable urinary catheters in resealable packaging using chlorine-containing species.

### BACKGROUND TO THE INVENTION

Urinary catheterisation is a process involving insertion of a catheter through an individual's urethra and into their bladder, where it is retained to empty the bladder of urine. There are two major types of urinary catheterisation-intermittent catheterisation and long-term catheterisation. Intermittent urinary catheterisation involves retaining the catheter in the bladder for only the time period required for emptying, after which the catheter is removed. The process differs from long-term catheterisation, which makes use of an indwelling or Foley catheter that is inserted into the bladder for long periods of time (several days to months) to discharge the residual urine of the bladder continuously throughout the day.

Catheterisation is often used by patients suffering from abnormalities of the urinary system, resulting in urinary incontinence and/or a lack of control in permitting voluntary urination. Such individuals would typically make use of intermittent catheters several times a day.

Catheters are useful devices, providing users with independence and freedom to self-catheterise as and when required, without having to rely on trained personnel to be present. This, however, increases the need for catheters to be user friendly: in particular, both easy to insert and remove with minimum discomfort caused, and safe to use with features for minimising risk of infection. Users often report experiencing pain and discomfort upon insertion and/or removal of catheters. Users have, for instance, reported experiencing bladder spasms, burning sensations, and bleeding.

It is also easy for catheters to become contaminated and for bacteria to be introduced into the urethra and along the urinary tract. As a result, urinary tract infections (UTI) are common in individuals who practice self-catheterisation.

Based on the above, urinary catheters, especially intermittent catheters, are typically single-use items. A user will remove the catheter from a package, use the catheter once, and then dispose of the catheter and package. This can be undesirable as it generates unwanted waste. Reusable urinary catheters could, thus, be advantageous is reducing the amount of waste created, but there are various challenges associated with the use of reusable catheters, such as issues surrounding storage, sterilisation and lubrication of the catheters, which need to be overcome before widespread acceptance and use of reusable catheters.

Sterilisation and lubrication of reusable catheters are two especially important factors, yet effective sterilisation and lubrication of reusable catheters has been notoriously difficult to achieve in practice and in most cases highly inconvenient to the catheter user.

There is a particular need for new solutions to the above challenges which allow for safe, effective and simple reuse of catheters.

It is an aim of embodiments of the present invention to address one or more of the above problems by providing a setup for reusable catheterisation which provides one or more of the following advantages:
Allows for simple and effective catheter sterilisation.
Allows for simple and effective catheter lubrication.
Allows for combined sterilisation and lubrication.
Allows for catheter sterilisation/lubrication without adversely impacting the catheter, preferably without adversely impacting the catheter surface properties.
Allows for the catheter to be reused safely for long time periods.
Does not require too frequent re-sterilisation and/or re-lubrication.
Allows for a reduced likelihood of infections c.f. reusable setups of the prior art.
Allows for a high lubricity, non-stick catheter surface to be maintained even after reuse. Allows for maintained ease of insertion and removal of the catheter even after reuse.
Allows for an inert and sterile catheter surface to be maintained even after reuse.
Allows for maintained catheter performance even after reuse.
Not overly complex setup and simple/cost-effective to implement.

It is also an aim of embodiments of the invention to overcome or mitigate at least one problem of the prior art, whether expressly described herein or not.

### SUMMARY OF THE INVENTION

According to a first aspect of the invention, there is provided a reusable urinary catheter kit comprising a container comprising: a urinary catheter; and a medium comprising at least one chlorine-containing species, wherein the container is resealable.

The medium comprising chlorine-containing species allows for effective sterilisation and/or lubrication of the catheter, which can be achieved by simply contacting the catheter with the medium in the container. This setup therefore allows for the catheter to be removed from the resealable container for use and then sterilised after use by simply reinserting the catheter back into the container and bringing the catheter into contact with the medium in the container. The catheter can then simply be removed from the container again for the next use without the need for any further sterilisation/lubrication steps.

The present inventors have also shown that this re-use/sterilisation cycle can be repeated multiple times with a single catheter and without the need to replace the medium between uses, and catheters are still safe to use and maintain their performance.

Further, use of such a medium causes minimal change to the relevant surface chemistry of the catheter upon contact, and so allows for catheter multi-use without adversely impacting catheter lubricity. The medium also provides for long-term sterilisation and/or lubrication, allowing the catheter to be reused safely for relatively long time periods, without the need for overly frequent re-sterilisation and/or re-lubrication.

In some embodiments, the catheter comprises a hollow tubular body, preferably a hollow polymeric tubular body. The hollow polymeric tubular body may comprise a base polymer.

In some embodiments, the catheter further comprises at least one additive, preferably at least one lubricious additive. Preferably, the hollow tubular body comprises at least one additive.

Additives allow for improved catheter lubricity and ease of insertion and removal. However, the use of additives on a catheter, particularly on a surface thereof, provides the catheter with complex surface chemistry which can make catheter sterilisation and repeated lubrication challenging. Such catheters have, for instance, been known to swell upon wetting during sterilisation. Such catheters are also known to suffer from dry-out, which can result in considerable changes to the catheter surface morphology. As a result, catheters can become rough and sticky, and much more prone to additive delamination. The above negatives are typically severely amplified with catheter reuse. However, a medium containing chlorine-containing species of the invention allows for effective catheter sterilisation and/or lubrication, which allows for prolonged effects of the additives. Accordingly, such a medium allows for safe and simple catheter use, with excellent performance in relation to reducing pain and discomfort.

In some embodiments, at least one additive is a hydrophilic additive.

At least one hydrophilic additive may be independently chosen from: a polyalkylene glycol, hyaluronic acid, chondroitan sulfate, chitosan, glucosaminoglucans, dextran, dextrin, dextran sulfate, cellulose acetate, carboxymethyl cellulose, hydroxyethyl cellulose, cellulosics, polypeptides, poly(2-hydroxyethyl methacrylate), polyacrylamide, polyacrylimide, poly(ethylene amine), poly(allyl amine), poly(vinyl pyrrolidone) (PVP), poly(vinyl alcohol), poly(acrylic acid), poly(methacrylic acid), acrylic acid copolymers, methacrylic acid copolymers, polyvinyl alkyl ethers, non-ionic tetrafunctional block-copolymer surfactants, gelatin, collagen, albumin, chitin, heparin, elastin, fibrin, and combinations thereof.

At least one hydrophilic additive may be independently selected from the group consisting of: a polyalkylene glycol, hyaluronic acid, chondroitan sulfate, chitosan, glucosaminoglucans, dextran, dextrin, dextran sulfate, cellulose acetate, carboxymethyl cellulose, hydroxyethyl cellulose, cellulosics, polypeptides, poly(2-hydroxyethyl methacrylate), polyacrylamide, polyacrylimide, poly(ethylene amine), poly(allyl amine), poly(vinyl pyrrolidone) (PVP), poly(vinyl alcohol), poly(acrylic acid), poly(methacrylic acid), acrylic acid copolymers, methacrylic acid copolymers, polyvinyl alkyl ethers, non-ionic tetrafunctional block-copolymer surfactants, gelatin, collagen, albumin, chitin, heparin, elastin, fibrin, and combinations thereof.

In some embodiments, at least one hydrophilic additive is independently chosen from: poly(ethylene glycol), poly(ethylene oxide), poly(propylene glycol), poly(ethylene oxide-co-propylene oxide), poly(trimethylene glycol), poly(tetramethylene glycol), and combinations thereof.

In some embodiments, at least one hydrophilic additive is independently selected from the group consisting of: poly(ethylene glycol), poly(ethylene oxide), poly(propylene glycol), poly(ethylene oxide-co-propylene oxide), poly(trimethylene glycol), poly(tetramethylene glycol), and combinations thereof.

At least one hydrophilic additive may comprise PVP or a derivative thereof.

At least one additive may be an amphiphilic additive. The amphiphilic additive comprises a hydrophobic portion and a hydrophilic portion. In cases where the base polymer is hydrophobic or generally hydrophobic, such as a polyolefin, the amphiphilic additive will diffuse towards and to an outer surface of the catheter body due to incompatibility of the hydrophilic portion of the amphiphilic additive with the hydrophobic base polymer.

Amphiphilic additives, in particular those described below, further allow a hydration layer to be created through temporary hydrogen bonds on the catheter surface with water molecules and the chlorine-containing species. As such, the catheter is able to remain sterile and lubricated for long periods of time and minimal to no changes to surface morphology are seen during sterilisation/lubrication and during catheter dry-out.

In some embodiments, the catheter comprises a hollow polymeric tubular body comprising a base polymer and an amphiphilic lubricious additive.

In some embodiments, at least one additive is polymeric or oligomeric.

At least one additive may be an A-B block copolymer comprising a hydrophobic hydrocarbon A-block and a hydrophilic B-block. In some embodiments, one or both of the hydrophobic hydrocarbon A-block and the hydrophilic B-block may be branched.

The hydrophobic A-block may comprise hydrophobic hydrocarbon chains branching therefrom. The hydrophobic hydrocarbon chains may be of shorter chain lengths than the hydrophobic hydrocarbon A-block. The hydrophilic B-block may comprise further hydrophilic B-blocks branching therefrom.

In some embodiments, the additive is a B-A-B tri-block copolymer comprising a hydrophobic hydrocarbon A-block and hydrophilic B-blocks.

In other embodiments, the additive is a graft copolymer. The graft copolymer may comprise a hydrophobic hydrocarbon A-block with hydrophilic B-blocks branching therefrom. Alternatively, the graft copolymer may comprise a hydrophilic portion with hydrophobic portions branching therefrom.

In further embodiments, the additive is a brush copolymer. The additive may comprise a single hydrophilic B-block with more than one hydrophobic A-block branching from an end thereof. Alternatively, the additive may comprise a single hydrophobic A-block with more than one hydrophilic B-block branching from an end thereof. In the respective embodiments, the B-block or A-block may comprise 2, 3, 4, 5, 6, 7, 8, 9, 10, or more hydrophobic A-blocks or hydrophilic B-blocks branching from the end thereof. In further embodiments, the additive is a star-block or multi-block copolymer comprising hydrophilic and hydrophobic monomer units.

In preferred embodiments, the additive is an A-B block copolymer comprising a hydrophobic A-block and a hydrophilic B-block.

Statements of invention below relating to the additive or a part thereof may be applied mutatis mutandis to each of the copolymer forms above.

In some embodiments, the B-block is a hydrophilic oligomer comprising at least 1, 2, 3, 4, or at least 5 monomer units. In some embodiments, the B-block comprises no greater than 20, 19, 18, 17, 16, 15, 14, 13, 12, 11, 10, 9, 8, 7, or no greater than 6 monomer units. In some embodiments, the B-block comprises between 2 and 15 monomer units, preferably between 2 and 10 monomer units. At least one monomer unit may be selected from the group consisting of: alkylene oxides, alkylene glycols, epihalohydrins, unsaturated carboxylic acids, alkylene imines, lactones, vinyl alcohol, and vinyl alkanoates. At least one monomer unit may be chosen from: alkylene oxides, alkylene glycols, epihalohydrins, unsaturated carboxylic acids, alkylene imines, lactones, vinyl alcohol, and vinyl alkanoates. At least one monomer unit may be preferably selected from the group consisting of: ethylene oxide, propylene oxide, ethylene glycol, propylene glycol, epichlorohydrin, acrylic acid, methacrylic acid, ethylene imine, caprolactone, vinyl alcohol, and vinyl acetate. At least one monomer unit may be preferably chosen from: ethylene oxide, propylene oxide, ethylene glycol, propylene glycol, epichlorohydrin, acrylic acid, methacrylic acid, ethylene imine, caprolactone, vinyl alcohol, and vinyl acetate. In some embodiments, at least one monomer unit comprises alkylene oxide groups independently selected from ethylene oxide and propylene oxide, and in preferred embodiments, all of the monomer units are ethylene oxide or all of the monomer units are propylene oxide.

The hydrophobic A-block may comprise a carbon chain of at least 5 carbon atoms, or at least 10, 15, 20, 25, 30, 35, or 40 carbon atoms. The hydrophobic portion may preferably comprise a carbon chain of between 20-52 carbon atoms.

In some embodiments, the A-block comprises a hydrocarbon chain block of the formula CH₃CH₂(CH₂CH₂)ₐ. The value of “a” may be between 5-25; for instance, “a” may be 5, 6, 7, 8, 9, 10, 11, 12, 13, 14, 15, 16, 17, 18, 19, 20, 21, 22, 23, 24, or 25, or a half integer of any of the above values. The value of “a” may preferably be between 9-25; for instance, “a” may be 9, 10, 11, 12, 13, 14, 15, 16, 17, 18, 19, 20, 21, 22, 23, 24, or 25, or a half integer of any of the above values.

In some embodiments, the additive is homogenously distributed with the polymer. The additive may be uniformly distributed throughout the base polymer of the catheter body.

At least some of the additive may be at or on the outer surface of the body. By “at the outer surface”, it is meant that at least a portion of the additive forms part of the surface or protrudes from the surface. In some embodiments, part of the additive is retained or anchored in the body while part of the additive forms part of or protrudes from the outer surface of the body. At least part of the hydrophilic portion of the additive may protrude from or form part of the outer surface of the body, while at least part of the hydrophobic portion may be retained or anchored within the body.

The outer surface may comprise at least one member of the group consisting of: the external-facing surface of the body, the lumen of the body and any eyelets present on the body. In preferred embodiments the outer surface is the external-facing surface of the body and/or the inner lumen. In some embodiments, the outer surface may comprise the external-facing surface of the body of the catheter, the inner lumen, and the eyelets.

The additive may be concentrated at or on the outer surface of the body. For example, at least 25%, 30%, 35%, 40%, 45%, 50%, 55%, 60%, 65%, 70%, 75%, 80%, 85%, 90%, or at least 95% of the number of molecules of the additive may be at or on the outer surface of the body.

In some embodiments, at least 25%, 30%, 35%, 40%, 45%, 50%, 55%, 60%, 65%, 70%, 75%, 80%, 85%, 90%, or at least 95% of the number of molecules of additive may have hydrophilic portions that are at or on the outer surface of the body.

In some embodiments, the additive is located at and/or on at least 50, 55, 60, 65, 70, 75, 80, 85, 90, 95, 96, 97, 98 or at least 99% of the outer surface area of the polymeric tubular body, preferably at least 75% or at least 90% of the outer surface area of the polymeric tubular body or between 75% and 100% of the outer surface area.

In some embodiments, the additive is present at a concentration of at least 0.1, 0.2, 0.3. 0.4. 0.5, 0.75, 1, 2, 3, 4, 5, 10, 15 or at least 20% by weight of the combination of base polymer and additive. The additive may be present a concentration of between 0.1-20%, and more preferably between 0.5-15% or 0.5-5% by weight of the combination of base polymer and additive.

In some embodiments, the additive comprises a layer that is on or that comprises a surface of the body, preferably the outer surface.

The layer comprising the additive may be on the surface of the body. In some embodiments, the layer comprising the additive is substantially separate from the body and the layer may be bonded to the body. The layer may be bonded to the body via covalent bonds, ionic bonds, hydrogen bonds, or Van der Waals forces. The additive may be bonded to the body via one or more surface linker groups which may be present on the additive, the body of the catheter or both.

In some embodiments, the layer comprising the additive may comprise the surface of the body. In such embodiments the layer may form the surface of the body. The layer may comprise a co-extruded layer which is melded with or is physically entangled with the body, and this may form an integral layer. The layer of additive may be integrally formed with the body.

In some embodiments, polymer diffusion occurs between the layer comprising the additive and the catheter body. The layer and the body may be held together by polymer chains extending across the interface between the layer and body. In some embodiments, the additive infiltrates the catheter body.

In some embodiments, the layer comprising the additive comprises or is on an inner surface of the body, an outer surface of the body, or both. The inner surface of the body may comprise a lumen of the catheter. In preferred embodiments, the layer comprising the additive comprises or is on at least an outer surface of the body.

In some embodiments, the layer comprising the additive is on or comprises at least 50, 55, 60, 65, 70, 75, 80, 85, 90, 95, 96, 97, 98 or at least 99% of the or each surface area of the body, preferably at least 75% or at least 90% of the or each surface area or between 75% and 100% of the or each surface area. In embodiments in which the layer comprising the additive comprises or is on both an inner and outer surface of the body, the additive may comprise at least 50, 55, 60, 65, 70, 75, 80, 85, 90, 95, 96, 97, 98 or at least 99% of each surface area of the body, preferably at least 75% or at least 90% of each surface area or between 75% and 100% of each surface area of both surfaces.

In some embodiments, at least 75% of the layer comprising the additive, or at least 76, 77, 78, 79, 80, 81, 82, 83, 84, 85, 86, 87, 88, 89, 90, 91, 92, 93, 94, 95, 96, 97, 98, 99, or 100% of the layer is the additive.

In some embodiments, the layer comprising the additive has an additive concentration of at least 0.1, 0.2, 0.3. 0.4. 0.5, 0.75, 1, 2, 3, 4, 5, 10, 15 or at least 20% by weight of the combination of base polymer and additive.

In some embodiments, the layer comprising the additive has an additive concentration of no greater than 70, 65, 60, 65, 60, 55, or of no greater than 50% by weight of the combination of the base polymer and additive.

The layer comprising the additive may have an additive concentration of greater than 5% by weight of the combination of the base polymer and additive. The layer may have an additive concentration of between 6-50% by weight of the combination of the base polymer and additive.

The layer comprising the additive may have an additive concentration of between 10-50% by weight of the combination of the base polymer and additive, or of between 15-50, 20-50, 25-50, 30-50, 35-50, 40-50, or of between 45-50% by weight of the combination of the base polymer and additive.

The layer comprising the additive may have an additive concentration of between 6-45% by weight of the combination of the base polymer and additive, or of between 6-40, 6-35, 6-30, 6-25, 6-20, 6-15, or of between 6-10% by weight of the combination of the base polymer and additive.

The layer comprising the additive may have an additive concentration of between 10-45% by weight of the combination of the base polymer and additive, or of between 15-45, 20-45, 25-45, 30-45, 35-45, 40-45, 10-40, 15-40, 20-40, 25-40, 30-40, 35-40, 10-35, 15-35, 20-35, 25-35, 30-35, 10-30, 15-30, 20-30, 25-30, 10-25, 15-25, 20-25, 10-20, 15-20, or of between 10-15% by weight of the combination of the base polymer and additive.

In some embodiments, the layer comprising the additive has a thickness of at least 1 μm, or of at least 2, 3, 4, 5, 6, 7, 8, 9, 10, 15, 20, 25, 30, 35, 40, 45, or of at least 50 μm.

In some embodiments, the layer comprising the additive has a thickness of no more than 10000 μm, or of no more than 9000, 8000, 7000, 6000, 5000, 4000, 3000, 2000, 1000, 900, 800, 700, 600, 500, 400, or of no more than 300 μm.

In some embodiments, the layer comprising the additive has a thickness of between 50-300 μm.

The layer comprising the additive may have a thickness of between 60-300 μm, or of between 80-300, 100-300, 120-300, 140-300, 160-300, 180-300, 200-300, 220-300, 240-300, 260-300, or of between 280-300 μm.

The layer comprising the additive may have a thickness of between 50-280 μm, or of between 50-260, 50-240, 50-220, 50-200, 50-180, 50-160, 50-140, 50-120, 50-100, 50-80, or of between 50-60 μm.

The layer comprising the additive may have a thickness of between 60-280 μm, or of between 80-280, 100-280, 120-280, 140-280, 160-280, 180-280, 200-280, 220-280, 240-280, 260-280, 60-260, 80-260, 100-260, 120-260, 140-260, 160-260, 180-260, 200-260, 220-260, 240-260, 60-240, 80-240, 100-240, 120-240, 140-240, 160-240, 180-240, 200-240, 220-240, 60-220, 80-220, 100-220, 120-220, 140-220, 160-220, 180-220, 200-220, 60-200, 80-200, 100-200, 120-200, 140-200, 160-200, 180-200, 60-180, 80-180, 100-180, 120-180, 140-180, 160-180, 60-160, 80-160, 100-160, 120-160, 140-160, 60-140, 80-140, 100-140, 120-140, 60-120, 80-120, 100-120, 60-100, 80-100, or of between 60-80 μm.

In preferred embodiments, the catheter base polymer is hydrophobic or partly hydrophobic. A hydrophobic base polymer facilitates increased hydrophobic-hydrophobic interactions between the hydrophobic portion of the additive and the base polymer. This further decreases the energetic favourability for the hydrophobic portion to leave the base polymer and migrate out into the more hydrophilic external environment.

In some embodiments, the base polymer comprises a polymer chosen from: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, styrene-butadiene copolymer (SBC), styrene-ethylene-butylene-styrene copolymer (SEBS), and water disintegrable or enzymatically hydrolysable material, or combinations, blends or copolymers of any of the above materials.

In some embodiments, the base polymer comprises a polymer selected from the group consisting of: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, styrene-butadiene copolymer (SBC), styrene-ethylene-butylene-styrene copolymer (SEBS), and water disintegrable or enzymatically hydrolysable material, or combinations, blends or copolymers of any of the above materials.

In preferred embodiments, the base polymer comprises a polymer chosen from: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or copolymers of any of the above materials.

In preferred embodiments, the base polymer comprises a polymer selected from the group consisting of: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or copolymers of any of the above materials.

In some embodiments, said water disintegrable or enzymatically hydrolysable material comprises a material chosen from: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

In some embodiments, said water disintegrable or enzymatically hydrolysable material comprises a material of the group consisting of: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

In other preferred embodiments, the base polymer comprises a polymer chosen from: polyolefins, polyvinyl chloride, polyurethane, styrene-butadiene copolymer (SBC), styrene-ethylene-butylene-styrene copolymer (SEBS), and thermoplastic elastomeric material or combinations, blends or copolymers of any of the above materials.

In other preferred embodiments, the base polymer comprises a polymer selected from the group consisting of: polyolefins, polyvinyl chloride, polyurethane, styrene-butadiene copolymer (SBC), styrene-ethylene-butylene-styrene copolymer (SEBS), and thermoplastic elastomeric material or combinations, blends or copolymers of any of the above materials.

In some preferred embodiments, the base polymer comprises a polyolefin, especially polyethylene and/or polypropylene.

In some preferred embodiments, the base polymer comprises a thermoplastic elastomeric material. The base polymer may comprise a thermoplastic polyolefin.

The thermoplastic base polymer may comprise a hydrophobic polymer chosen from: Accurel™, Styroflex™, Styrolux™, MelifleX™, and Mediprene™ and any combination thereof.

The thermoplastic base polymer may comprise a hydrophobic polymer selected from the group consisting of: Accurel™, Styroflex™, Styrolux™, MelifleX™, and Mediprene™ and any combination thereof.

The thermoplastic base polymer may comprise Estane™ 58315, which is both hydrophobic and hydrophilic.

In preferred embodiments, the catheter is an intermittent urinary catheter. Such a catheter is typically inserted into a body for short time periods, such as less than a day. Alternatively, the catheter may be an indwelling (Foley) catheter. Such a catheter is typically inserted and kept in a body for long periods of time, such as several days to months.

In some embodiments, the medium comprises at least one chlorine-containing species that is independently chosen from: hypochlorous acid, at least one hypochlorite salt, chlorine dioxide, molecular chlorine, a dichloroisocyanurate salt, chloroazodin, dichlorodimethylhydantoin, chloroxylenol, chlorhexidine, and combinations thereof.

In some embodiments, there is provided a reusable urinary catheter kit comprising a container comprising: a urinary catheter; and a medium comprising at least one chlorine-containing species that is independently chosen from: hypochlorous acid, at least one hypochlorite salt, chlorine dioxide, molecular chlorine, a dichloroisocyanurate salt, chloroazodin, dichlorodimethylhydantoin, chloroxylenol, chlorhexidine, and combinations thereof, and wherein the container is resealable. In some embodiments, the medium comprises at least one chlorine-containing species that is independently selected from the group consisting of: hypochlorous acid, at least one hypochlorite salt, chlorine dioxide, molecular chlorine, a dichloroisocyanurate salt, chloroazodin, dichlorodimethylhydantoin, chloroxylenol, chlorhexidine, and combinations thereof.

In preferred embodiments, the medium comprises at least one chlorine-containing species that is independently chosen from: hypochlorous acid, at least one hypochlorite salt, chlorine dioxide, and combinations thereof.

In some preferred embodiments, there is provided a reusable urinary catheter kit comprising a container comprising: a urinary catheter; and a medium comprising at least one chlorine-containing species that is independently chosen from: hypochlorous acid, at least one hypochlorite salt, chlorine dioxide, and combinations thereof, and wherein the container is resealable.

In preferred embodiments, the medium comprises at least one chlorine-containing species that is independently selected from the group consisting of: hypochlorous acid, at least one hypochlorite salt, chlorine dioxide, and combinations thereof.

In some preferred embodiments, the medium comprises at least one chlorine-containing species independently chosen from: hypochlorous acid, chlorine dioxide, and combinations thereof.

In some preferred embodiments, there is provided a reusable urinary catheter kit comprising a container comprising: a urinary catheter; and a medium comprising at least one chlorine-containing species that is independently chosen from: hypochlorous acid, chlorine dioxide, and combinations thereof, and wherein the container is resealable.

In some embodiments, the medium comprises chlorine dioxide.

In some embodiments, the medium comprises hypochlorous acid. In some preferred embodiments, the medium comprises hypochlorous acid and at least one further chlorine-containing species.

In some preferred embodiments, there is provided a reusable urinary catheter kit comprising a container comprising: a urinary catheter; and a medium comprising hypochlorous acid and at least one further chlorine-containing species, and wherein the container is resealable.

In some embodiments, the medium comprises at least one hypochlorite salt. At least one hypochlorite salt may preferably comprise a countercation. The countercation may comprise an inorganic countercation. The countercation may comprise a metal countercation. In some embodiments, at least one countercation is independently chosen from: an alkali metal cation, an alkaline earth metal cation, a group III metal cation, a transition metal cation, an ammonium cation, an aromatic nitrogen-based cation, and combinations thereof. In some embodiments, at least one countercation is independently selected from the group consisting of: an alkali metal cation, an alkaline earth metal cation, a group III metal cation, a transition metal cation, an ammonium cation, an aromatic nitrogen-based cation, and combinations thereof. At least one countercation may be independently chosen from: ammonium, calcium, iron, magnesium, potassium, pyridinium, quaternary ammonium, sodium, copper, aluminium, lithium, beryllium, strontium, and zinc. At least one countercation may be independently selected from the group consisting of: ammonium, calcium, iron, magnesium, potassium, pyridinium, quaternary ammonium, sodium, copper, aluminium, lithium, beryllium, strontium, and zinc. At least one countercation may preferably be an alkali metal cation or an alkaline earth metal cation. At least one countercation may preferably be independently chosen from: calcium, lithium, and sodium. In preferred embodiments, at least one hypochlorite salt is sodium hypochlorite. At least one countercation may preferably be independently selected from the group consisting of: calcium, lithium, and sodium. In preferred embodiments, at least one hypochlorite salt is sodium hypochlorite.

In some embodiments, the medium comprises hypochlorous acid and at least one hypochlorite salt. At least one hypochlorite salt may preferably be as described in statements of invention above. The medium may preferably comprise hypochlorous acid and at least one alkali metal hypochlorite salt. In some embodiments, the medium comprises hypochlorous acid and sodium hypochlorite.

In some preferred embodiments, there is provided a reusable urinary catheter kit comprising a container comprising: a urinary catheter; and a medium comprising hypochlorous acid and at least one hypochlorite salt, and wherein the container is resealable.

In embodiments wherein the medium comprises hypochlorous acid and at least one hypochlorite salt, the pH of the medium may preferably be between 6-9.

In some embodiments, the medium comprises chlorine dioxide and at least one of: hypochlorous acid and at least one hypochlorite salt. In some embodiments, the medium comprises chlorine dioxide, hypochlorous acid, and at least one hypochlorite salt. In such embodiments, at least one hypochlorite salt may preferably be as described in statements of invention above. At least one hypochlorite salt may preferably be an alkali metal hypochlorite salt, which may comprise sodium hypochlorite.

In some embodiments, the medium is present in liquid, gel or solid form.

In some embodiments, the medium is present as a solution of the chlorine-containing species in a solvent. The solution may be an aqueous solution. Aqueous solutions are particularly effective, as water allows for optimal catheter surface lubricity. The solvent may therefore be water or an aqueous medium.

In some embodiments, the medium comprises the chlorine-containing species in a total concentration of between 0.005-1.5 wt. % of the medium, or between 0.005-1, or between 0.005-0.6 wt. % of the medium, or between 0.01-0.5, 0.015-0.4, 0.02-0.3, or between 0.025-0.2 wt. % of the medium, or preferably between 0.025-0.1 wt. % of the medium.

In some embodiments, the medium comprises the chlorine-containing species in a total concentration of at least 1 ppm, or at least 2, 3, 4, 5, 6, 7, 8, 9, or at least 10 ppm. In some embodiments, the medium comprises the chlorine-containing species in a total concentration of no greater than 100 ppm, or no greater than 90, 80, 70, 60, or no greater than 50 ppm. In some embodiments, the medium comprises the chlorine-containing species in a total concentration of between 1-100 ppm, or between 5-100, 10-100, 10-90, 10-80, 10-70, 10-60, or between 10-50 ppm.

In some preferred embodiments, there is provided a reusable urinary catheter kit comprising a container comprising: a urinary catheter; and a medium comprising at least one chlorine-containing species, wherein the container is resealable, and wherein the at least one chlorine-containing species is present in the medium in a total concentration of between 1-100 ppm.

In some embodiments, the medium comprises chlorine dioxide in a total concentration of at least 0.0001 wt. % of the medium, or at least 0.0002, 0.0003, 0.0004, 0.0005, 0.0006, 0.0007, 0.0008, 0.0009, 0.001, 0.002, 0.003, 0.004, or at least 0.005 wt. % of the medium, or at least 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the medium.

In some embodiments, the medium comprises chlorine dioxide in a total combined concentration of no greater than 5 wt. % of the medium, or no greater than 4.5, 4, 3.5, 3, 2.5, 2, 1.5, 1, 0.9, 0.8, 0.7, 0.6, 0.5, 0.4, 0.3, or no greater than 0.2 wt. % of the medium, or no greater than 0.1, 0.05, 0.04, 0.03, 0.02, 0.01, 0.009, 0.008, 0.007, 0.006, 0.005, 0.004, 0.003, 0.002, or no greater than 0.001 wt. % of the medium.

In some embodiments, the medium comprises chlorine dioxide in a total combined concentration of between 0.005-1.5 wt. % of the medium, or between 0.005-1, or between 0.005-0.6 wt. % of the medium, or between 0.01-0.5, 0.015-0.4, 0.02-0.3, or between 0.025-0.2 wt. % of the medium, or preferably between 0.025-0.1 wt. % of the medium.

In some embodiments, the medium comprises chlorine dioxide in a total concentration of at least 1 ppm, or at least 2, 3, 4, 5, 6, 7, 8, 9, or at least 10 ppm. In some embodiments, the medium comprises chlorine dioxide in a total concentration of no greater than 100 ppm, or no greater than 90, 80, 70, 60, or no greater than 50 ppm. In some embodiments, the medium comprises chlorine dioxide in a total concentration of between 1-100 ppm, or between 5-100, 10-100, 10-90, 10-80, 10-70, 10-60, or between 10-50 ppm.

In some preferred embodiments, there is provided a reusable urinary catheter kit comprising a container comprising: a urinary catheter; and a medium comprising at least one chlorine-containing species comprising chlorine dioxide, wherein the container is resealable, and wherein the medium comprises chlorine dioxide in a total concentration of between 1-100 ppm.

In some embodiments, the medium comprises one or more of: chlorine dioxide, hypochlorous acid, and at least one hypochlorite in a total combined concentration of at least 0.0001 wt. % of the medium, or at least 0.0002, 0.0003, 0.0004, 0.0005, 0.0006, 0.0007, 0.0008, 0.0009, 0.001, 0.002, 0.003, 0.004, or at least 0.005 wt. % of the medium, or at least 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the medium.

In some embodiments, the medium comprises one or more of: chlorine dioxide, hypochlorous acid, and at least one hypochlorite salt in a total combined concentration of no greater than 5 wt. % of the medium, or no greater than 4.5, 4, 3.5, 3, 2.5, 2, 1.5, 1, 0.9, 0.8, 0.7, 0.6, 0.5, 0.4, 0.3, or no greater than 0.2 wt. % of the medium, or no greater than 0.1, 0.05, 0.04, 0.03, 0.02, 0.01, 0.009, 0.008, 0.007, 0.006, 0.005, 0.004, 0.003, 0.002, or no greater than 0.001 wt. % of the medium.

In some embodiments, the medium comprises one or more of: chlorine dioxide, hypochlorous acid, and at least one hypochlorite salt in a total combined concentration of between 0.005-1.5 wt. % of the medium, or between 0.005-1, or between 0.005-0.6 wt. % of the medium, or between 0.01-0.5, 0.015-0.4, 0.02-0.3, or between 0.025-0.2 wt. % of the medium, or preferably between 0.025-0.1 wt. % of the medium.

In some embodiments, the medium comprises one or more of: chlorine dioxide, hypochlorous acid, and at least one hypochlorite salt in a total combined concentration of at least 1 ppm, or at least 2, 3, 4, 5, 6, 7, 8, 9, or at least 10 ppm. In some embodiments, the medium comprises one or more of: chlorine dioxide, hypochlorous acid, and at least one hypochlorite salt in a total combined concentration of no greater than 100 ppm, or no greater than 90, 80, 70, 60, or no greater than 50 ppm. In some embodiments, the medium comprises one or more of: chlorine dioxide, hypochlorous acid, and at least one hypochlorite salt in a total combined concentration of between 1-100 ppm, or between 5-100, 10-100, 10-90, 10-80, 10-70, 10-60, or between 10-50 ppm.

In some preferred embodiments, there is provided a reusable urinary catheter kit comprising a container comprising: a urinary catheter; and a medium comprising at least one chlorine-containing species, wherein the container is resealable, and wherein the medium comprises one or more of: chlorine dioxide, hypochlorous acid, and at least one hypochlorite salt in a total combined concentration of between 1-100 ppm.

In some embodiments, the medium comprises hypochlorous acid and/or at least one hypochlorite in a total combined concentration of at least 0.0001 wt. % of the medium, or at least 0.0002, 0.0003, 0.0004, 0.0005, 0.0006, 0.0007, 0.0008, 0.0009, 0.001, 0.002, 0.003, 0.004, or at least 0.005 wt. % of the medium, or at least 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the medium.

In some embodiments, the medium comprises hypochlorous acid and/or at least one hypochlorite salt in a total combined concentration of no greater than 5 wt. % of the medium, or no greater than 4.5, 4, 3.5, 3, 2.5, 2, 1.5, 1, 0.9, 0.8, 0.7, 0.6, 0.5, 0.4, 0.3, or no greater than 0.2 wt. % of the medium, or no greater than 0.1, 0.05, 0.04, 0.03, 0.02, 0.01, 0.009, 0.008, 0.007, 0.006, 0.005, 0.004, 0.003, 0.002, or no greater than 0.001 wt. % of the medium.

In some embodiments, the medium comprises hypochlorous acid and/or at least one hypochlorite salt in a total combined concentration of between 0.005-1.5 wt. % of the medium, or between 0.005-1, or between 0.005-0.6 wt. % of the medium, or between 0.01-0.5, 0.015-0.4, 0.02-0.3, or between 0.025-0.2 wt. % of the medium, or preferably between 0.025-0.1 wt. % of the medium.

In some embodiments, the medium comprises hypochlorous acid and/or at least one hypochlorite salt in a total combined concentration of at least 1 ppm, or at least 2, 3, 4, 5, 6, 7, 8, 9, or at least 10 ppm. In some embodiments, the medium comprises hypochlorous acid and/or at least one hypochlorite salt in a total combined concentration of no greater than 100 ppm, or no greater than 90, 80, 70, 60, or no greater than 50 ppm. In some embodiments, the medium comprises hypochlorous acid and/or at least one hypochlorite salt in a total combined concentration of between 1-100 ppm, or between 5-100, 10-100, 10-90, 10-80, 10-70, 10-60, or between 10-50 ppm.

In some preferred embodiments, there is provided a reusable urinary catheter kit comprising a container comprising: a urinary catheter; and a medium comprising at least one chlorine-containing species comprising hypochlorous acid and/or at least one hypochlorite salt, wherein the container is resealable, and wherein the hypochlorous acid and/or at least one hypochlorite salt is present in a total combined concentration of between 1-100 ppm of the medium.

In some embodiments, the medium comprises hypochlorous acid in a total concentration of at least 0.0001 wt. % of the medium, or at least 0.0002, 0.0003, 0.0004, 0.0005, 0.0006, 0.0007, 0.0008, 0.0009, 0.001, 0.002, 0.003, 0.004, or at least 0.005 wt. % of the medium, or at least 0.006, 0.007, 0.008, 0.009, 0.01, 0.015, 0.02, 0.025, 0.03, 0.035, 0.04, 0.045, 0.05, 0.055, 0.06, or preferably at least 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the medium.

In some embodiments, the medium comprises hypochlorous acid in a total concentration of no greater than 5 wt. % of the medium, or no greater than 4.5, 4, 3.5, 3, 2.5, 2, 1.5, 1, 0.9, 0.8, 0.7, 0.6, 0.5, 0.4, 0.3, or no greater than 0.2 wt. % of the medium, or no greater than 0.1, 0.05, 0.04, 0.03, 0.02, 0.01, 0.009, 0.008, 0.007, 0.006, 0.005, 0.004, 0.003, 0.002, or no greater than 0.001 wt. % of the medium.

In some embodiments, the medium comprises hypochlorous acid in a total concentration of between 0.005-1.5 wt. % of the medium, or between 0.005-1, or between 0.005-0.6 wt. % of the medium, or between 0.01-0.5, 0.015-0.4, 0.02-0.3, or between 0.025-0.2 wt. % of the medium, or between 0.025-0.1 wt. % of the medium.

In some embodiments, the medium comprises hypochlorous acid in a total concentration of at least 1 ppm, or at least 2, 3, 4, 5, 6, 7, 8, 9, or at least 10 ppm. In some embodiments, the medium comprises hypochlorous acid in a total concentration of no greater than 100 ppm, or no greater than 90, 80, 70, 60, or no greater than 50 ppm. In some embodiments, the medium comprises hypochlorous acid in a total concentration of between 1-100 ppm, or between 5-100, 10-100, 10-90, 10-80, 10-70, 10-60, or between 10-50 ppm.

In some preferred embodiments, there is provided a reusable urinary catheter kit comprising a container comprising: a urinary catheter; and a medium comprising at least one chlorine-containing species comprising hypochlorous acid, wherein the container is resealable, and wherein the medium comprises the hypochlorous acid in a total concentration of between 1-100 ppm.

The medium may comprise the hypochlorite salt in a total concentration of at least 0.0001 wt. % of the medium, or at least 0.0002, 0.0003, 0.0004, 0.0005, 0.0006, 0.0007, 0.0008, 0.0009, 0.001, 0.002, 0.003, 0.004, or at least 0.005 wt. % of the medium, or at least 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the medium.

In some embodiments, the medium comprises the hypochlorite salt in a total concentration of no greater than 5 wt. % of the medium, or no greater than 4.5, 4, 3.5, 3, 2.5, 2, 1.5, 1, 0.9, 0.8, 0.7, 0.6, 0.5, 0.4, 0.3, or no greater than 0.2 wt. % of the medium, or no greater than 0.1, 0.05, 0.04, 0.03, 0.02, 0.01, 0.009, 0.008, 0.007, 0.006, 0.005, 0.004, 0.003, 0.002, or no greater than 0.001 wt. % of the medium.

In some embodiments, the medium comprises the hypochlorite salt in a total concentration of between 0.005-1.5 wt. % of the medium, or between 0.005-1, or between 0.005-0.6 wt. % of the medium, or between 0.01-0.5, 0.015-0.4, 0.02-0.3, or between 0.025-0.2 wt. % of the medium, or between 0.025-0.1 wt. % of the medium.

In some embodiments, the medium comprises at least one hypochlorite salt in a total concentration of at least 1 ppm, or at least 2, 3, 4, 5, 6, 7, 8, 9, or at least 10 ppm. In some embodiments, the medium comprises at least one hypochlorite salt in a total concentration of no greater than 100 ppm, or no greater than 90, 80, 70, 60, or no greater than 50 ppm. In some embodiments, the medium comprises at least one hypochlorite salt in a total concentration of between 1-100 ppm, or between 5-100, 10-100, 10-90, 10-80, 10-70, 10-60, or between 10-50 ppm.

In some preferred embodiments, there is provided a reusable urinary catheter kit comprising a container comprising: a urinary catheter; and a medium comprising at least one chlorine-containing species comprising at least one hypochlorite salt, wherein the container is resealable, and wherein the medium comprises the at least one hypochlorite salt in a total concentration of between 1-100 ppm.

In some embodiments, the medium is present in a total amount of at least 0.05 wt. % of the combination of the catheter and medium, or of at least 0.1, 0.15, 0.2, 0.25, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9, 10, 15, or at least 20 wt. % of the combination of the catheter and medium.

The medium may be present in a total amount of no greater than 40 wt. % of the combination of the catheter and medium, or no greater than 35, 30, 25, 20, 15, 10, or no greater than 5 wt. % of the combination of the catheter and medium.

The medium may be present in a total amount of between 0.1-20 wt. % of the combination of the catheter and medium, or between 0.5-15, or between 0.5-5 wt. % of the combination of the catheter and medium.

In some embodiments, the medium is present as a liquid having a viscosity of greater than 0.5 cP, or of greater than 1, 1.5, 2, 3, 4, 5, 10, 20, 30, 40, 50, 75, 100, 150, 200, 300, 400, 500, 600, 700, 800, 900, or of greater than 1000 cP. The medium may have a viscosity of no greater than 100000 cP, or of no greater than 90000, 80000, 70000, 60000, 50000, 40000, 30000, 20000, 10000, 5000, 4000, 3000, 2000, 1000, 500, 400, 300, 200, 100, 50, 25, 10, 5, 4, 3, 2, or of no greater than 1 cP. The medium may have a viscosity of between 0.5-5000 cP, or of between 0.5-2500, 0.5-1000, 1-1000, 10-1000, 50-1000, 100-1000, 500-1000, 0.5-500, 1-500, 10-500, 50-500, 100-500, 250-500, 0.5-250, 1-250, 10-250, 50-250, or of between 100-250 cP.

The medium may be a catheter wetting agent. The medium may encourage hydrophilic portions of lubricating additives within the catheter to seek towards an outer surface of the catheter, which further enhances the lubricating effect of the additive.

In some embodiments, the medium has a pH of at least 2, 2.5, 3, 3.5, 4, 4.5, 5, 5.5, 6, 6.5, 7, 7.5, 8, 8.5, or of no greater than 9. In some embodiments, the medium may have a pH of no greater than 14, or of no greater than 13.5, 13, 12.5, 12, 11.5, 11, 10.5, or of no greater than 10.

In some embodiments, the medium has a pH of between 2-14, 3-13, or of between 4-12.

In some embodiments, the medium comprises at least one further species that is independently chosen from: a peroxide species, a base, an acid, a photosensitiser, a permanganate species, an alcohol, a phenol, an aldehyde, ionic silver, molecular iodine or an iodophor, an imine-containing species, a salt and combinations thereof.

In some embodiments, the medium comprises at least one further species that is independently selected from the group consisting of: a peroxide species, a base, an acid, a photosensitiser, a permanganate species, an alcohol, a phenol, an aldehyde, ionic silver, molecular iodine or an iodophor, an imine-containing species, a salt and combinations thereof.

In preferred embodiments, the medium comprises molecular chlorine. The medium may comprise molecular chlorine and at least one species independently chosen from: hypochlorous acid, at least one hypochlorite salt, chlorine dioxide, and combinations thereof. The medium may comprise molecular chlorine and at least one of: hypochlorous acid and a hypochlorite salt. In some embodiments, the medium comprises at least one hypochlorite salt and molecular chlorine. In preferred embodiments, the medium comprises hypochlorous acid and molecular chlorine. In some embodiments, the medium comprises hypochlorous acid, molecular chlorine, and at least one hypochlorite salt.

In some preferred embodiments, there is provided a reusable urinary catheter kit comprising a container comprising: a urinary catheter; and a medium comprising at least one chlorine-containing species, wherein the container is resealable, and wherein the at least one chlorine-containing species comprises molecular chlorine and further comprises at least one species independently chosen from: hypochlorous acid, at least one hypochlorite salt, chlorine dioxide, and combinations thereof.

In some preferred embodiments, there is provided a reusable urinary catheter kit comprising a container comprising: a urinary catheter; and a medium comprising at least one chlorine-containing species, wherein the container is resealable, and wherein the at least one chlorine-containing species comprises molecular chlorine and further comprises hypochlorous acid.

In some embodiments, the medium has a pH of less than 4, and the amount of molecular chlorine in the medium is greater than the total combined amount of hypochlorous acid and hypochlorite salt.

In some embodiments, the medium has a pH of between 4-7, and the amount of hypochlorous acid in the medium is greater than the total combined amount of molecular chlorine and hypochlorite salt.

In some embodiments, the medium has a pH of greater than 8, and the amount of hypochlorite salt in the medium is greater than the total combined amount of molecular chlorine and hypochlorous acid.

The medium may comprise at least one further salt (in addition to any hypochlorite salt present). At least one salt may comprise a cation that is independently chosen from: ammonium, calcium, iron, magnesium, potassium, pyridinium, quaternary ammonium, sodium, copper, aluminium, lithium, beryllium, strontium, and zinc. At least one salt may comprise a cation that is independently selected from the group consisting of: ammonium, calcium, iron, magnesium, potassium, pyridinium, quaternary ammonium, sodium, copper, aluminium, lithium, beryllium, strontium, and zinc. At least one salt may preferably comprise an alkali metal cation. At least one salt may comprise an anion that is independently chosen from: acetate, carbonate, bicarbonate, chloride, citrate, glutamate, fluoride, bromide, iodide, nitrate, nitrite, oxide, phosphate, ferrocyanide, silicate, gluconate, and sulfate. At least one salt may comprise an anion that is independently selected from the group consisting of: acetate, carbonate, bicarbonate, chloride, citrate, glutamate, fluoride, bromide, iodide, nitrate, nitrite, oxide, phosphate, ferrocyanide, silicate, gluconate, and sulfate. At least one salt may preferably comprise a halide anion.

At least one of the further salts may be independently chosen from: sodium chloride, potassium chloride, calcium chloride, magnesium chloride, calcium chloride, sodium nitrite, magnesium nitrate, calcium nitrate, potassium carbonate, sodium carbonate, sodium bicarbonate, potassium iodide, copper iodide, sodium ferrocyanide, monosodium glutamate, calcium silicate, sodium citrate, potassium citrate, sodium phosphate, potassium phosphate, sodium sulfate, calcium sulfate, sodium gluconate, calcium gluconate, potassium gluconate, sodium acetate, and potassium acetate.

At least one of the further salts may be independently selected from the group consisting of: sodium chloride, potassium chloride, calcium chloride, magnesium chloride, calcium chloride, sodium nitrite, magnesium nitrate, calcium nitrate, potassium carbonate, sodium carbonate, sodium bicarbonate, potassium iodide, copper iodide, sodium ferrocyanide, monosodium glutamate, calcium silicate, sodium citrate, potassium citrate, sodium phosphate, potassium phosphate, sodium sulfate, calcium sulfate, sodium gluconate, calcium gluconate, potassium gluconate, sodium acetate, and potassium acetate.

At least one of the further salts may preferably be an alkali metal halide. At least one salt may be independently chosen from: an alkali metal chloride, an alkali metal bromide, an alkali metal iodide, and combinations thereof. At least one salt may be independently selected from the group consisting of: an alkali metal chloride, an alkali metal bromide, an alkali metal iodide, and combinations thereof.

Preferably, the medium may comprise an alkali metal salt and a hypochlorite salt. The medium may comprise a halide salt and a hypochlorite salt. The medium may comprise an alkali metal halide and a hypochlorite salt.

In a particularly preferred embodiment, the medium comprises sodium chloride. The medium may comprise sodium and at least one of: hypochlorous acid and a hypochlorite salt. The medium may preferably comprise a hypochlorite salt and sodium chloride, preferably an alkali metal hypochlorite and sodium chloride, and more preferably sodium hypochlorite and sodium chloride.

In some embodiments, the total concentration of further salt in the medium is between 5-30 wt. %, or of between 10-25, or of between 10-20 wt. % of the medium.

In some embodiments, the medium comprises the hypochlorite salt in a total concentration of between 0.25-2 wt. %, or 0.5-1.5 wt. % of the medium, and the further salt in a total concentration of between 5-30, 10-25, or between 10-20 wt. % of the medium.

In some embodiments, the ratio of the total concentration of further salt to hypochlorite salt in the medium is at least 5, or at least 10, or at least 15. The ratio of the total concentration of further salt to hypochlorite salt in the medium may be between 5-30, or between 10-25, or preferably between 10-20.

In some preferred embodiments, there is provided a reusable urinary catheter kit comprising a container comprising: a urinary catheter; and a medium comprising at least one chlorine-containing species comprising at least one hypochlorite salt, wherein the container is resealable, and wherein the medium further comprises at least one alkali metal halide salt, preferably comprising sodium chloride, wherein the ratio of the total concentration of the at least one alkali metal halide salt to the at least one hypochlorite salt in the medium is between 10-20.

In some embodiments, the medium further comprises at least one base. The medium may comprise a base and at least one chlorine-containing species chosen from:

hypochlorous acid, at least one hypochlorite salt, chlorine dioxide, and combinations thereof. The medium may comprise a base and at least one of: hypochlorous acid and a hypochlorite salt. In preferred embodiments, the medium may comprise a base and a hypochlorite salt.

At least one base may comprise an inorganic base. At least one inorganic base may be independently chosen from: a hydroxide base, a carbonate base, a bicarbonate base, and combinations thereof. At least one inorganic base may be independently selected from the group consisting of: a hydroxide base, a carbonate base, a bicarbonate base, and combinations thereof. In some preferred embodiments, at least one base is a hydroxide base. At least one hydroxide base may be independently chosen from: an alkali metal hydroxide, an alkaline earth metal hydroxide, a group III metal hydroxide, a transition metal hydroxide, and combinations thereof. At least one hydroxide base may be independently selected from the group consisting of: an alkali metal hydroxide, an alkaline earth metal hydroxide, a group III metal hydroxide, a transition metal hydroxide, and combinations thereof. At least one hydroxide base may preferably comprise an alkali metal hydroxide and/or an alkaline earth metal hydroxide. In some embodiments, at least one base comprises an alkali metal hydroxide that is independently chosen from: lithium hydroxide, sodium hydroxide, potassium hydroxide, and combinations thereof. In some embodiments, at least one base comprises an alkali metal hydroxide that is independently selected from the group consisting of: lithium hydroxide, sodium hydroxide, potassium hydroxide, and combinations thereof.

In some preferred embodiments, there is provided a reusable urinary catheter kit comprising a container comprising: a urinary catheter; and a medium comprising at least one chlorine-containing species independently chosen from: hypochlorous acid, at least one hypochlorite salt, chlorine dioxide, and combinations thereof, wherein the container is resealable, and wherein the medium further comprises at least one base, preferably at least one alkali metal hydroxide base, preferably comprising sodium hydroxide.

Preferably, the medium may comprise an alkali metal base and a hypochlorite salt. The medium may comprise a hydroxide base and a hypochlorite salt. The medium may comprise an alkali metal hydroxide and a hypochlorite salt.

In a particularly preferred embodiment, the base comprises sodium hydroxide. The medium may comprise sodium hydroxide and at least one of: hypochlorous acid and a hypochlorite salt. The medium may preferably comprise a hypochlorite salt and sodium hydroxide, preferably an alkali metal hypochlorite and sodium hydroxide, and more preferably sodium hypochlorite and sodium hydroxide.

In some preferred embodiments, there is provided a reusable urinary catheter kit comprising a container comprising: a urinary catheter; and a medium comprising at least one chlorine-containing species comprising at least one hypochlorite salt, wherein the container is resealable, and wherein the medium further comprises at least one base, preferably at least one alkali metal hydroxide base, preferably comprising sodium hydroxide.

In some embodiments, the medium comprises at least one acid that is a carboxylic acid. In some embodiments, the medium comprises at least one acid that is independently chosen from: acetic acid, citric acid, peracetic acid, diperoxy dodecanoic acid, and combinations thereof. In some embodiments, the medium comprises at least one acid that is independently selected from the group consisting of: acetic acid, citric acid, peracetic acid, diperoxy dodecanoic acid, and combinations thereof.

In some embodiments, the medium comprises at least one C1-C10 alcohol, or at least one C1-C5 alcohol, or at least one C1-C3 alcohol. In some embodiments, the medium comprises at least one alcohol that is independently chosen from: ethanol, isopropanol, and combinations thereof. In some embodiments, the medium comprises at least one alcohol that is independently selected from the group consisting of: ethanol, isopropanol, and combinations thereof.

In some embodiments, the medium comprises at least one phenol that is independently chosen from: phenol, thymol, chloroxylenol, and combinations thereof.

In some embodiments, the medium comprises at least one phenol that is independently selected from the group consisting of: phenol, thymol, chloroxylenol, and combinations thereof.

In some embodiments, the medium comprises at least one aldehyde that is independently chosen from: glutaraldehyde, noxytiolin, and combinations thereof.

In some embodiments, the medium comprises at least one aldehyde that is independently selected from the group consisting of: glutaraldehyde, noxytiolin, and combinations thereof.

In some embodiments, the medium comprises at least one imine-containing species that is independently selected from: polyhexanide, octenidine, and combinations thereof.

The container preferably comprises a resealable opening. The resealable opening may be configured such that the catheter can be removed and inserted through the resealable opening. The resealable opening may be opened and closed using a resealable opening mechanism that is independently chosen from: a zipper closure, a press-to-seal closure, a resealable lid, a peel-and-seal closure, a slider closure, and a Velcro closure. The resealable opening may be opened and closed using a resealable opening mechanism that is independently selected from the group consisting of: a zipper closure, a press-to-seal closure, a resealable lid, a peel-and-seal closure, a slider closure, and a Velcro closure.

The container may preferably define a cavity configured to hold the catheter, and preferably comprise a resealable opening. The catheter may preferably be configured to be repeatedly inserted and removed from the container, preferably through the resealable opening thereof.

In some embodiments, the container is rigid. In other embodiments, the container is flexible. In some embodiments, the container contains rigid and flexible regions. The container may comprise a tube. The tube may have a flexible centre portion and two rigid side portions neighbouring the flexible centre portion. The tube may be resealable at a side portion thereof.

In some embodiments, the container is a pouch comprising a resealable opening. The catheter may preferably be configured to be repeatedly inserted and removed from the pouch, preferably through the resealable opening thereof.

The catheter may preferably be configured to be repeatedly inserted and removed from the container, preferably through the resealable opening thereof.

In some embodiments, the medium is in direct contact with the catheter. The medium may be in direct contact with at least one surface of the catheter. The at least one surface may comprise the outer surface of the catheter. The medium may cover at least part of the outer surface of the catheter.

The at least one surface may comprise an inner surface of the catheter and the medium may cover at least part of the inner surface.

In some embodiments, the medium is in direct contact with the inner and outer surfaces of the catheter.

In some embodiments, the catheter is in direct contact with the medium along at least 30% of the catheter length, or at least 40, 50, 60, 70, 80, 90, or at least 95% of the catheter length. In some embodiments, the catheter is in direct contact with the medium along the full length of the catheter.

In some embodiments, at least the outer surface of the catheter, and preferably both the inner and outer surfaces of the catheter are in direct contact with the medium along at least 30% of the catheter length, or at least 40, 50, 60, 70, 80, 90, or at least 95% of the catheter length. In some embodiments, at least the outer surface of the catheter, and preferably both the inner and outer surfaces of the catheter are in direct contact with the medium along the full length of the catheter.

In some embodiments, the catheter is submerged in the medium. Submersion of the catheter in the medium has been shown to confer optimal catheter lubricity.

The catheter may be fully submerged in the medium. In such embodiments, the medium may comprise at least 30% of the internal volume of the container, or at least 40, 50, 60, 70, 80, 90, or at least 95% of the internal volume of the container.

In some embodiments, the catheter is not fully submerged in the medium. In such embodiments, the medium may comprise no greater than 30% of the internal volume of the container, or no greater than 25, 20, 15, 10, 5, 4, 3, 2, or no greater than 1% of the internal volume of the container. In such embodiments, the medium may be in direct contact with at least part of the catheter, preferably with at least one surface thereof. In some embodiments, the medium may be able to move freely within the container. In such embodiments, the medium may be configured to slosh around within the container, such as when the container is shaken and/or due to natural movement of the container by the user.

In some embodiments, the catheter comprises a layer of the medium that is on or that comprises at least part of a surface of the catheter, preferably at least part of the outer surface of the catheter. In such embodiments, the medium may be a liquid or a gel.

The layer of medium may preferably be on the surface of the catheter, preferably on the surface of the catheter body.

In some embodiments, the layer is on an inner surface of the catheter, an outer surface of the catheter, or both. The inner surface of the catheter may comprise a lumen of the catheter. In preferred embodiments, the layer is on at least an outer surface of the catheter.

In some embodiments, the layer is on at least 50, 55, 60, 65, 70, 75, 80, 85, 90, 95, 96, 97, 98, or at least 99% of the or each surface area of the catheter, preferably at least 75%, or at least 90% of the or each surface area, or between 75% and 100% of the or each surface area. In embodiments in which the layer is on both an inner and outer surface of the catheter, the additive may be on or comprise at least 50, 55, 60, 65, 70, 75, 80, 85, 90, 95, 96, 97, 98 or at least 99% of each surface area of the catheter, preferably at least 75% or at least 90% of each surface area or between 75% and 100% of each surface area of both surfaces.

In some embodiments, at least 75% of the layer, or at least 76, 77, 78, 79, 80, 81, 82, 83, 84, 85, 86, 87, 88, 89, 90, 91, 92, 93, 94, 95, 96, 97, 98, 99, or 100% of the layer is the medium.

In some embodiments, the layer has a thickness of at least 1 μm, or of at least 2, 3, 4, 5, 6, 7, 8, 9, 10, 15, 20, 25, 30, 35, 40, 45, or of at least 50 μm.

In some embodiments, the layer has a thickness of no greater than 10000 μm, or of no greater than 9000, 8000, 7000, 6000, 5000, 4000, 3000, 2000, 1000, 900, 800, 700, 600, 500, 400, or of no greater than 300 μm.

In some embodiments, the layer has a thickness of between 50-300 μm.

The layer may have a thickness of between 60-300 μm, or of between 80-300, 100-300, 120-300, 140-300, 160-300, 180-300, 200-300, 220-300, 240-300, 260-300, or of between 280-300 μm.

The layer may have a thickness of between 50-280 μm, or of between 50-260, 50-240, 50-220, 50-200, 50-180, 50-160, 50-140, 50-120, 50-100, 50-80, or of between 50-60 μm.

The layer may have a thickness of between 60-280 μm, or of between 80-280, 100-280, 120-280, 140-280, 160-280, 180-280, 200-280, 220-280, 240-280, 260-280, 60-260, 80-260, 100-260, 120-260, 140-260, 160-260, 180-260, 200-260, 220-260, 240-260, 60-240, 80-240, 100-240, 120-240, 140-240, 160-240, 180-240, 200-240, 220-240, 60-220, 80-220, 100-220, 120-220, 140-220, 160-220, 180-220, 200-220, 60-200, 80-200, 100-200, 120-200, 140-200, 160-200, 180-200, 60-180, 80-180, 100-180, 120-180, 140-180, 160-180, 60-160, 80-160, 100-160, 120-160, 140-160, 60-140, 80-140, 100-140, 120-140, 60-120, 80-120, 100-120, 60-100, 80-100, or of between 60-80 μm.

The layer of medium may have a viscosity of at least 200 cP, or at least 400, 600, 800, or at least 1000 cP, or at least 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, 9000, 9200, 9400, 9600, 9800, or at least 10000 cP.

The layer of medium may have a viscosity of no greater than 2000000, or no greater than 1750000, 1500000, 1250000, or no greater than 1000000 cP.

The layer of medium may have a viscosity of between 100-2000000, or between 500-1000000, or between 1000-500000 cP.

In some embodiments, the medium is contained in a medium container and the catheter is contained in a separate catheter container. The medium container may be a bag or sachet. The medium container may be located in the catheter container. In some embodiments, the medium is contained in a medium container located in the catheter container and the medium is not in direct contact with the catheter. In some embodiments, the medium container is rupturable or pierceable, in use, to release the contained medium from the medium container and into direct contact with the catheter in the catheter container, preferably without requiring opening of the catheter container.

Prior to opening the catheter container and/or prior to removing the catheter, the user may release the medium from the medium container to bring the medium into direct contact with the catheter in the catheter container, preferably to bring the medium into direct contact with the outer surface of the catheter.

In some embodiments, the medium container is configured such that when the catheter container is opened, the contained medium is released from the separate medium container and into direct contact with the catheter. The medium container may be configured to rupture or break to release the contained medium from the separate medium container and into direct contact with the catheter upon opening of the catheter container.

In some embodiments, the catheter is submerged in a liquid and/or solution which does not contain the chlorine-containing species, and the chlorine-containing species may be contained in a separate medium container. The separate medium container may be as described in statements of invention above.

The chlorine-containing species may be present in the separate medium container in solid, liquid, or gel form. The liquid may comprise a solution comprising the chlorine-containing species. In embodiments wherein the chlorine-containing species are present in solid form, the solid may comprise a powder or at least one tablet.

In such embodiments, the chlorine-containing species may be releasable from the separate medium container, in use. The chlorine-containing species of the invention may be used to treat the catheter after release from the medium container. In embodiments wherein the chlorine-containing species is present in solid form, the solid may be dissolvable in a liquid to provide a solution comprising the chlorine-containing species, which may be used to treat the catheter. The liquid may be the liquid in which the catheter is submerged.

In some embodiments, the reusable catheter may be configured to be reused at least 1 time, or at least 2, 3, 4, 5, 6, 7, 8, 9, 10, 11, 12, 13, 14, 15, 16, 17, 18, 19, or at least 20 times. The reusable catheter may be configured to be reused up to 5 times, or up to 10, 15, 20, 25, 30, 35, 40, 45, or up to 50 times.

In some embodiments, the reusable catheter may be configured to be reused for up to 10 hours, or up to 15, or up to 20 hours, preferably up to 1 day, or up to 2, 3, 4, 5, or up to 6 days, or up to 1 week, or up to 8 days, or up to 9, 10, 11, 12, or up to 13 days, or up to 2 weeks, or up to 3 weeks, or up to 1 month, or up to 2 months. The reusable catheter may be configured to be reused for up to between 1 day to 2 month, or up to between 1 week to 1.5 months, or for up to between 2 weeks to 1 month.

In some embodiments, the reusable catheter is configured to be reused without needing to replenish and/or change the medium. In some embodiments, the reusable catheter is configured to be reused for up to 5 hours without needing to replenish and/or change the medium, or for up to 10 hours, or for up to 15, or 20, or for up to 24 hours without needing to replenish and/or change the medium.

According to a second aspect of the invention, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species;
(b) Unsealing the container;
(c) Removing the urinary catheter from the unsealed container;
(d) Performing catheterisation with the catheter;
(e) Reinserting the catheter into the container and contacting the catheter with the medium comprising at least one chlorine-containing species in the container; and
(f) Resealing the container.

The catheter of the second aspect of the invention is preferably the catheter of the first aspect of the invention. The medium of the second aspect of the invention is preferably the medium of the first aspect of the invention. The container of the second aspect of the invention is preferably the container of the first aspect of the invention.

Statements of invention above relating to the first aspect of the invention may also be applied mutatis mutandis to the second aspect of the invention.

In some embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species that is independently chosen from: hypochlorous acid, at least one hypochlorite salt, chlorine dioxide, molecular chlorine, a dichloroisocyanurate salt, chloroazodin, dichlorodimethylhydantoin, chloroxylenol, chlorhexidine, and combinations thereof;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species that is independently chosen from: hypochlorous acid, at least one hypochlorite salt, chlorine dioxide, and combinations thereof;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species that is independently chosen from: hypochlorous acid, chlorine dioxide, and combinations thereof;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising hypochlorous acid and at least one further chlorine-containing species;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising hypochlorous acid and at least one hypochlorite salt;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species, wherein the at least one chlorine-containing species is present in the medium in a total concentration of between 1-100 ppm;
(b) Unsealing the container;
(c) Removing the urinary catheter from the unsealed container;
(d) Performing catheterisation with the catheter;
(e) Reinserting the catheter into the container and contacting the catheter with the medium comprising at least one chlorine-containing species in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species comprising chlorine dioxide, wherein the medium comprises chlorine dioxide in a total concentration of between 1-100 ppm;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising one or more of: chlorine dioxide, hypochlorous acid, and at least one hypochlorite salt in a total combined concentration of between 1-100 ppm;
(b) Unsealing the container;
(c) Removing the urinary catheter from the unsealed container;
(d) Performing catheterisation with the catheter;
(e) Reinserting the catheter into the container and contacting the catheter with the medium; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species comprising hypochlorous acid and/or at least one hypochlorite salt, wherein the hypochlorous acid and/or at least one hypochlorite salt is present in a total combined concentration of between 1-100 ppm of the medium;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species comprising hypochlorous acid, wherein the medium comprises the hypochlorous acid in a total concentration of between 1-100 ppm;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species comprising at least one hypochlorite salt, wherein the medium comprises the at least one hypochlorite salt in a total concentration of between 1-100 ppm;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species comprising molecular chlorine and further comprising at least one species independently chosen from: hypochlorous acid, at least one hypochlorite salt, chlorine dioxide, and combinations thereof;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species comprising molecular chlorine and further comprising hypochlorous acid;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species comprising at least one hypochlorite salt, wherein the medium further comprises at least one alkali metal halide salt, preferably comprising sodium chloride, wherein the ratio of the total concentration of the at least one alkali metal halide salt to the at least one hypochlorite salt in the medium is between 10-20;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species independently chosen from: hypochlorous acid, at least one hypochlorite salt, chlorine dioxide, and combinations thereof, and wherein the medium further comprises at least one base, preferably at least one alkali metal hydroxide base, preferably comprising sodium hydroxide;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some preferred embodiments, there is provided a method of sterilising and/or lubricating a reusable urinary catheter, the method comprising the steps of:
(a) Providing a resealable container in its sealed configuration, said container comprising a urinary catheter, and further providing a medium comprising at least one chlorine-containing species comprising at least one hypochlorite salt, and wherein the medium further comprises at least one base, preferably at least one alkali metal hydroxide base, preferably comprising sodium hydroxide;
(b) Unsealing the container;
(c) Removing the urinary catheter from the unsealed container;
(d) Performing catheterisation with the catheter;
(e) Reinserting the catheter into the container and contacting the catheter with the medium in the container; and
(f) Resealing the container.

In some embodiments, the medium is contained in the container in step (a) of the method. The catheter may be in direct contact with the medium in the container in step (a), preferably as described in statements of invention for the first aspect of the invention above. At least one surface of the catheter may be in direct contact with the medium in the container in step (a). The catheter may be submerged in the medium in step (a).

In some embodiments, the medium is contained in a separate medium container that is located in the catheter container in step (a) and the medium is not in direct contact with the catheter. The separate medium container may preferably be as described in statements of invention for the first aspect of the invention above. The method may further comprise the step of releasing the contained medium from the separate medium container and into direct contact with the catheter. The step of releasing the contained medium may be performed between steps (a) and (b) of the method. The step of releasing the contained medium may comprise piercing the medium container to release the contained medium from the medium container and into direct contact with the catheter in the catheter container.

In some embodiments, unsealing the catheter container in step (b) causes contained medium to be released from the separate medium container and into direct contact with the catheter in the catheter container. In some embodiments, unsealing the catheter container in step (b) causes the medium container to rupture or break to release the contained medium from the separate medium container and into direct contact with the catheter in the catheter container.

In some embodiments, the medium is not contained in the container in step (a) of the method, and the method comprises the further step of adding the medium into the container. The step of adding the medium into the container may comprise filling the container with the medium.

In some embodiments, the method further comprises the step of adding the medium into the container after step (b). The method may comprise the step of adding the medium into the container after step (b) and before reinserting the catheter into the container in step (e).

In some embodiments, the method further comprises the step of adding the medium into the container after reinserting the catheter into the container in step (e).

In some embodiments, step (e) comprises contacting at least one surface of the catheter with the medium, preferably at least an outer surface of the catheter.

In some embodiments, step (e) comprises contacting at least 20% of the outer surface area of the catheter with the medium, or at least 25, 30, 35, 40, 45, 50, 55, 60, 65, 70, 75, 80, 85, 90, 95, 96, 97, 98, or at least 99% of the outer surface area of the catheter, preferably at least 75%, or at least 90% of the outer surface area, or between 75% and 100% of the outer surface area of the catheter.

In some embodiments, step (e) comprises submerging the catheter in the medium.

Submerging the catheter in the medium, especially between uses, has been shown to result in optimal catheter lubricity.

In some embodiments, step (e) comprises contacting the catheter with the medium in the container for a total time of at least 5 seconds, or at least 10, 20, 30, 40, or at least 50 seconds, or at least 1 minute, or at least 2, 3, 4, or at least 5 minutes, or at least 10, 20, 30, 40, or at least 50 minutes, or at least 1 hour, or at least 1.5, 2, 2.5, 3, 3.5, or at least 4 hours. Step (e) may comprise contacting the catheter with the medium for a total time of no greater than 1 week, or no greater than 6 days, or no greater than 5, 4, 3, 2, or no greater than 1 day, or no greater than 20 hours, 19, 18, 17, 16, 15, 14, 13, 12, 11, 10, 9, 8, 7, 6, or no greater than 5 hours.

In some embodiments, step (e) is performed at a temperature of at least 1° C., or at least 2, 3, 4, or at least 5° C. In some embodiments, step (e) is performed at a temperature of no greater than 60° C., or no greater than 55, 50, 45, 40, 35, 30, or no greater than 25° C. In some embodiments, step (e) is performed at a temperature of between 1-60° C., or between 5-50, or between 10-40° C.

In some embodiments, the container in step (a) is provided before first use of the catheter. In some embodiments, the container in step (a) is provided after first use of the catheter. Steps (b) to (f) may be repeated. The method may comprise repeating steps (b) to (f) for each subsequent use of the catheter. In some embodiments, step (a) may comprise providing the container comprising an unused catheter, followed by initial steps (b) to (f), followed by a repetition of steps (b) to (f) for a subsequent use of the catheter. Such repetition of steps (b) to (f) may be completed a plurality of times. Steps (b) to (f) may be repeated at least 1, 2, 3, 4, 5, 6, or at least 7 times, preferably over a 24-hour period. Steps (b) to (f) may be repeated between 1-10 times, or between 1-7 times, preferably over a 24-hour period.

In some embodiments, the catheter is used for at least 5 hours after first use, or at least 10, 15, 20, or at least 24 hours after first use. In some embodiments, the catheter is used for up to 24 hours after first use.

There is no requirement to replenish or replace the medium contained in the catheter container between uses of the catheter. In embodiments wherein steps (b) to (f) are repeated, a repeated step (e) may comprise reinserting the catheter into the container and contacting the catheter with the same medium in the container as from the previous step (e), without the need to replenish or replace the medium between runs.

In some embodiments, the medium may not be replaced or replenished for at least 6 hours after the first catheter use, or at least 12, 18, or for at least 24 hours after the first catheter use.

In some embodiments, the medium may be replaced or replenished 18 hours or more after the first catheter use, or 20 hours or more, or 22 hours or more, or 24 hours or more after the first catheter use.

In some embodiments, the method further comprises a step of washing and/or rinsing the catheter before reinserting the catheter into the container in step (e). The method may comprise such a washing and/or rinsing step before each step (e) of the method. In some embodiments, the washing and/or rinsing step takes place directly before reinserting the catheter into the container in step (e). The washing and/or rinsing step may comprise washing and/or rinsing a surface of the catheter, preferably the outer surface thereof. In some embodiments, the washing and/or rinsing step comprises washing and/or rinsing the catheter with water. In some embodiments, the washing and/or rinsing step comprises washing and/or rinsing the catheter with an aqueous solution. The washing and/or rinsing step may comprise washing and/or rinsing the catheter with a detergent and with water or an aqueous solution. In some embodiments, the washing and/or rinsing step comprises applying a detergent to the catheter, preferably to the outer surface thereof, and then rinsing the catheter with water or an aqueous solution.

In some embodiments, the method comprises a further step of lubricating the catheter with an additional lubricating agent. The further lubrication step may preferably be performed after step (c). In some embodiments, the catheter may be dried after step (c) and before the further lubrication step. The drying step may comprise air drying and/or wiping the catheter to dry the catheter. In some embodiments, the further lubrication step is performed before use of the catheter, preferably directly before use of the catheter. Preferably, the further lubrication step is performed after step (c) and before use of the catheter.

The additional lubricating agent may be water or may comprise water. The additional lubricating agent may be an aqueous solution.

The further lubrication step may comprise treating the catheter, preferably at least part of the outer surface thereof with the lubricating agent. The further lubrication step may comprise applying the lubricating agent to the catheter, preferably to at least part of the outer surface of the catheter.

In some embodiments, a further lubrication step is not performed. In such embodiments, the catheter may be used directly after step (c). Treating the catheter with the medium comprising the chlorine-containing species provides both catheter sterilisation and lubrication and allows for optimal catheter lubrication even in the absence of a further lubrication step.

The following statements may be applied mutatis mutandis to all aspects of the present invention.

Catheter assemblies or components thereof disclosed in one or more of the following applications, which are hereby incorporated by reference in their entirety, may be used in the kit of the first aspect of the invention and/or the method of the second aspect of the invention: WO2019/123004A1, WO2019/123003A1, WO2022/223986A1, WO2022/223987A1, WO2022/223985A1, WO2022/223984A1, WO2022/223983A1, WO2022/223982A1, WO2022/223981A1, WO2022/223980A1, WO2022/223979A1, WO2022/223978A1, PCT/GB2023/052464, PCT/GB2023/052467, PCT/GB2023/052469, PCT/GB2023/052470, PCT/GB2023/052693, GB2305009.9, GB2314379.5, GB2314382.9, GB2314381.1, and PCT/GB2023/052465. In particular, the chemistry of the present invention may be used with the catheter assemblies or components thereof disclosed in the examples and figures of the above applications, or with any other devices/technologies as set out in the statements, examples and/or claims of the above applications.

Catheters, kits and components thereof marketed under the ConvaTec GentleCath

(RTM) product range, including catheter kits sold under the GentleCath (RTM) Glide and GentleCath (RTM) Glide Hydrophilic ranges (comprising elongated packaging containing a catheter and burstable sachets containing a wetting agent), may be used in the kit of the first aspect of the invention and/or the method of the second aspect of the invention. In particular, the chemistry of the present invention may be used with the catheters, kits and components thereof marketed under the GentleCath (RTM) range. For instance, the medium of the invention may be used as a wetting agent with the catheter kits sold under the GentleCath (RTM) Glide and GentleCath (RTM) Glide Hydrophilic ranges.

Flip open catheter packaging or components thereof disclosed in Hollister Inc EP patent application 3445436 and U.S. Pat. Nos. 10,561,817, 11,534,573 and 11,103,676, which are hereby incorporated by reference in their entirety, may be used in the kit of the first aspect of the invention and/or the method of the second aspect of the invention. In particular, the chemistry of the present invention may be used with the flip open catheter packaging or components thereof disclosed in the examples and figures of the above application and patents, or with any other devices/technologies as set out in the statements, examples and/or claims of the above application and patents.

Catheters, kits and components thereof marketed under the SpeediCath (RTM) product range by Coloplast Ltd, including catheter kits sold under the SpeediCath (RTM) Standard range (comprising elongated packaging containing a catheter and burstable sachets containing a wetting agent, or comprising a packaging containing a catheter in direct contact with a wetting agent), may be used in the kit of the first aspect of the invention and/or the method of the second aspect of the invention. In particular, the chemistry of the present invention may be used with the catheters, kits and components thereof marketed under the SpeediCath (RTM) range. For instance, the medium of the invention may be used as a wetting agent with the catheter kits sold under the SpeediCath (RTM) Standard range.

### DETAILED DESCRIPTION OF THE INVENTION

In order that the invention may be more clearly understood, embodiments thereof will now be described, by way of example only, with reference to the accompanying drawings, of which:

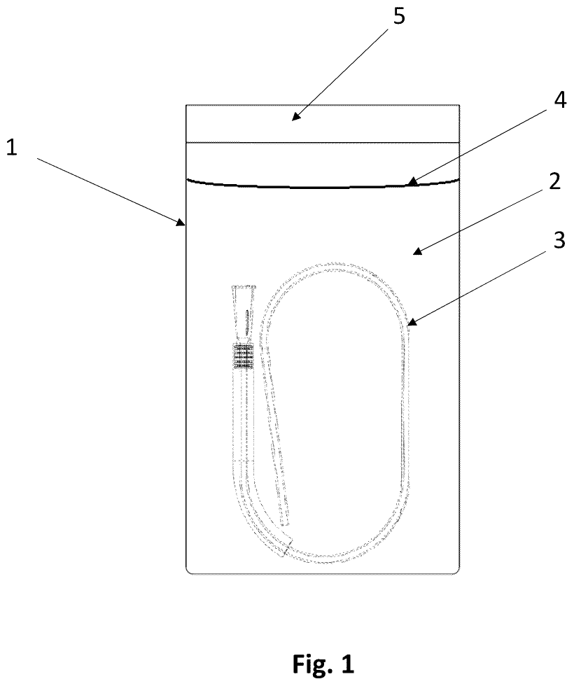

FIG. 1 is a front view of an embodiment of a reusable urinary catheter kit of the invention showing the resealable container in its sealed configuration.

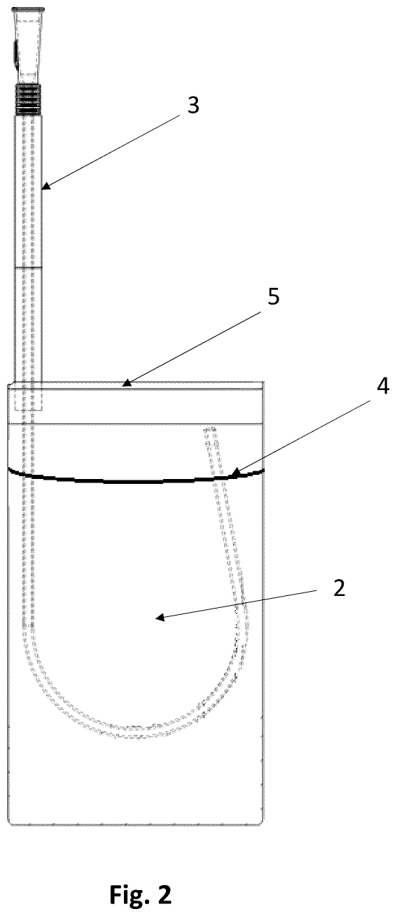

FIG. 2 is a front view of the reusable urinary catheter kit of FIG. 1 showing the resealable container in an open configuration.

### REUSABLE URINARY CATHETER KIT

FIGS. 1 and 2 illustrate an embodiment of a reusable urinary catheter kit of the invention. The kit comprises a resealable container 1 in the form of a catheter pouch 1. Pouch 1 defines an internal cavity 2 that is configured to hold a urinary catheter 3. The urinary catheter 3 is an intermittent catheter 3 comprising a hollow polymeric tubular body comprising a base polymer formed from a thermoplastic elastomeric material and further comprising an amphiphilic additive. The pouch 1 is also filled with a medium 4 comprising at least one chlorine-containing species-in this example, the medium 4 contains sodium hypochlorite and sodium chloride. The pouch 1 further comprises a resealable opening 5 formed at an edge of the pouch 1, which allows the pouch 1 to be repeatedly opened and closed. The resealable opening 5 comprises a resealable opening mechanism in the form of a press-to-seal closure. Such resealable opening mechanisms are known to those skilled in the art, who would also appreciate that in other embodiments, a range of alternative opening mechanisms may be employed.

In the sealed configuration of the pouch 1, as depicted in FIG. 1, the resealable opening 5 is in its sealed configuration. The catheter 3 is contained in the internal cavity 2 of the pouch 1 and is fully submerged in the medium 4 within the pouch 1. Both outer and inner surfaces (not shown) of the catheter 3 are in direct contact with the medium 4 along the full length of the catheter 3. In other embodiments, the medium 4 may be contained in a separate medium container that may be configured to release the medium 4 into the cavity 2 of the pouch to bring the catheter 3 into direct contact with the medium 4, before or upon first opening of the pouch.

In use, the user opens the pouch 1 at the resealable opening 5 and removes the catheter 3 from the pouch 1 through the resealable opening 5, as depicted in FIG. 2. The catheter 3 is then used in the conventional manner, without any requirement to rinse the catheter 3 after removal from the medium 4 and before use. After use, the catheter 3 is simply reinserted into the pouch 1 through the resealable opening 5, thus re-submerging the catheter 3 in the medium 4, and the pouch 1 resealed at the resealable opening 5. No rinsing/washing steps are necessary after use of the catheter 3, as reinserting the catheter 3 into the medium 4 effectively sterilises the catheter 3. However, it would also be possible for the user to rinse/wash the catheter 3 after use, depending on individual preference.

The catheter 3 is then re-used simply by removing the catheter 3 from the resealable pouch 1, as described, and re-using. Again, no further rinse/wash steps are required.

The catheter 3 was re-used in this manner with no additional wash/rinse steps performed and without replacing/replenishing the medium 4 in the pouch for 24 hours. Catheter lubricity was excellent even after such a long time period and even without medium replacement. The medium further provided excellent antimicrobial performance and overall catheter sterility. These variables are evaluated through the laboratory tests described below.

### Example 1—Catheter Lubrication Using Sodium Hypochlorite and Sodium Chloride Solution (No Solution Replenishment)

At room temperature, water was added to an empty catheter storage container. A tablet comprising sodium hypochlorite and sodium chloride was thereafter dissolved in the water to provide a medium of the invention containing sodium hypochlorite and sodium chloride.

Catheters, as described in Example 1 above, were submerged in an aqueous solution comprising sodium hypochlorite and sodium chloride. The catheters were left submerged in the solution for different periods of time (2, 4, 6, 8, and 24 hours) before their lubricity was tested by determination of their coefficient of friction (COF).

One sample set was tested immediately after removal from the aqueous solution. The second sample set included a rinse step with water to remove residual aqueous solution prior to catheter COF testing. A control set was also included where the catheter was submerged in pure water instead of the aqueous solution.

A sample size of n=10 was applied to each sample set. Combined COF data was generated for all timepoints.

COF values for catheters submerged in the sodium hypochlorite/sodium chloride solution were low and well within acceptable ranges for intermittent catheters with lubricating coatings. Further, the results showed that storage in the sodium hypochlorite/sodium chloride solution with no rinse step following removal provides the optimal catheter lubricity.

### Example 2—Catheter Sterilisation Using Sodium Hypochlorite and Sodium Chloride Solution

To assess the sterilisation potential of the medium comprising chlorine-containing species, a microbiology assessment was conducted which consisted of rate of kill assessments against repeat inoculation of E. coli and E. faecalis. These organisms were selected as they are commonly identified in catheter associated urinary tract infections. The selection also covers both gram-positive and gram-negative species to test the broad spectrum antimicrobial performance of the medium.

Intermittent urinary catheters, as used for Example 1, were provided and submerged in an aqueous medium comprising sodium hypochlorite and sodium chloride. The media comprising the catheters were respectively inoculated with the two microorganisms.

A direct inoculation method was applied against each microorganism. The test organisms were reinoculated following 1, 2, 3, 4, 5, 6, and 24 hours. Following each inoculation, a sample was taken after 5 seconds, 1, 5, 10 and 15 minutes to evaluate the rate of kill.

Rapid kill against E. coli and E. faecalis was observed up to 3 and 2 hours respectively. In the case of E. coli considerable efficacy was also observed after 15 minutes of inoculation for samples taken up to 6 hours after initial submersion of the catheter in the medium.

Overall, the results demonstrated excellent antimicrobial activity and sterilisation potential of the medium. Further, results show that the medium still provides significant antimicrobial activity even up to several hours after initial contact of the catheter with the medium.

### Example 3—Catheter Lubrication Using Hypochlorous Acid

Lubricity assessments analogous to those of Example 1 were performed—however, an aqueous hypochlorous acid solution was used as the medium in which the catheter was submerged between COF tests.

Similar results were observed as for Example 1, with COF values being achieved which were well within acceptable ranges for intermittent catheters with lubricating coatings.

Overall, performance in these tests demonstrated the lubricity enhancing effect of the hypochlorous acid containing medium of the invention, which was effective even after long time periods and without needing to replenish the medium between runs.

### Example 4—Catheter Lubrication Using Chlorine Dioxide

Lubricity assessments analogous to those of Example 3 were performed—however, an aqueous chlorine dioxide solution was used as the medium in which the catheter was submerged between COF tests.

Similar results were observed as for Example 3.

Overall, performance in these tests demonstrated the lubricity enhancing of the chlorine dioxide containing medium, which was similarly effective even after long time periods and without a need to replenish medium between runs.

### Example 5—Catheter Sterilisation Using Hypochlorous Acid

A microbiology assessment was performed using a medium of the invention comprising hypochlorous acid in an analogous manner to that performed for Example 2. However, for this test, following each inoculation, a sample was only taken after 5 seconds to evaluate the rate of kill. Further, in addition to the previous microorganisms tested, a further gram-positive organism, S. aureus, was tested.

The results demonstrated excellent antimicrobial activity, with rapid kill being observed within just 5 seconds following inoculation against S. aureus and E. faecalis for up to 6 hours and for up to 24 hours against E. coli.

To evaluate the antimicrobial activity further, the test was repeated using the same frequency of inoculation, but with sampling performed up to 5 minutes after reinoculation at 24 hours. Even for the most challenging organism, E. faecalis, a reduction in the microbial colony forming unit (CFU) counts of over 3 orders of magnitude was observed after 5 minutes of inoculation.

Overall, the results demonstrated excellent antimicrobial activity and sterilisation potential of the hypochlorous acid medium.

### Example 6—Catheter Sterilisation Using Chlorine Dioxide

A microbiology assessment was performed using a medium comprising chlorine dioxide in an analogous manner to that performed for Example 5.

The results demonstrated excellent antimicrobial activity, with rapid kill again being observed within just 5 seconds following inoculation against S. aureus and E. faecalis for up to 6 hours and for up to 24 hours against E. coli.

To evaluate the antimicrobial activity further, the test was repeated using the same frequency of inoculation, but with sampling performed up to 5 minutes after reinoculation at 24 hours. For the most challenging organism, E. faecalis, a reduction in the microbial colony forming unit (CFU) counts of 4 orders of magnitude was observed after around just 30 seconds of inoculation.

Overall, the results demonstrated excellent antimicrobial activity and sterilisation potential of the chlorine dioxide medium.

### Example 7—Effect of Hypochlorous Acid Concentration on Sterilisation Potential

The effect of the concentration of hypochlorous acid in the medium on sterilisation potential was tested. Aqueous media with hypochlorous acid concentrations of 250, 675, 1000, and 2000 ppm were supplied.

A microbiology assessment was performed using the media comprising hypochlorous acid in an analogous manner to that performed for Example 2. However, for this test, following each inoculation, samples were taken after 5 seconds, 30 seconds, 1, 2, and 5 minutes to evaluate the rate of kill.

Effective kill was found up to 24 hours after 5 minutes of sampling for each concentration of hypochlorous acid tested and for both challenging organisms tested, E. coli and E. faecalis.

Effective kill was also observed up to 24 hours for both organisms 30 seconds after sampling for a hypochlorous acid concentration of just 675 ppm.

For E. coli, highly effective kill was observed up to 5 hours for all time periods post-inoculation when using only a 250 ppm hypochlorous acid solution. For concentrations of 675 ppm and above, highly effective kill was observed up to 24 hours for all time periods post-inoculation.

For the more challenging organism, E. faecalis, highly effective kill was observed up to 4 hours for all time periods post-inoculation when using only a 250 ppm hypochlorous acid solution. Up to 5 hours, highly effective kill was observed just 30 seconds post-inoculation using the 250 ppm solution. At 675 ppm, highly effective kill was observed up to 6 hours for all time periods post-inoculation, and up to 24 hours from just 30 seconds post-inoculation. At 1000 ppm, highly effective kill was observed up to 24 hours for all time periods post-inoculation.

Overall, the results demonstrate that whilst antimicrobial activities of the media show dependence on the active ingredient concentration, excellent performance is shown at even very low concentrations and after long periods of time.

### Example 8—Effect of Very Low Hypochlorous Acid Concentration on Sterilisation Potential

The antimicrobial efficacy of hypochlorous acid was tested at even lower concentrations (10, 25, and 50 ppm) against the challenging organism E. coli NCIMB 14067.

The representative colonies of E. coli NCIMB 14067 were dispersed in maximum recovery diluent (MRD) to obtain an optical density (OD₅₄₀ₙₘ) equivalent to approximately 1×10⁶ CFU/mL. A quantitative plate count was performed on this suspension to determine the total number of viable organisms inoculated. A new suspension was prepared for each concentration tested.

A 1000 ppm hypochlorous acid solution was diluted in sterile deionised water to provide the respective 10, 25 and 50 ppm hypochlorous acid solutions.

The hypochlorous acid samples were aliquoted into 9 mL volumes and 1 mL of the challenge organism suspension was added and mixed (the final number of bacteria present is now approximately 1×10⁵ CFU/mL). Following 5, 30 seconds, 1, 2, and 5 minutes, 100 μL volumes were sampled and transferred to 0.9 mL and 9.9 mL of Dey-Engley Neutralising Broth (DENB) (to provide a 1:10 and 1:100 dilution respectively), and total viable counts were performed to establish the amount present. The plates were left to dry before being inverted and incubated at 35±3° C. for at least 48 hours. A negative control was also tested whereby 1 mL of the challenge organism suspension was added to 9 mL of MRD and tested as previously described. Following the incubation period, the number of CFUs were counted on the most appropriate dilution for each sample i.e. between 25-250 CFU/plate.

The results from each concentration of hypochlorous acid solution against E. coli NCIMB 14067 showed that 25 and 50 ppm hypochlorous acid solutions resulted in undetectable numbers of bacteria after 30 seconds. The 10 ppm hypochlorous acid solution did show some minor bacterial recovery at 30 seconds (to a little over 10 CFU/mL), but this was reduced to undetectable levels at the 1 minute timepoint. Bacterial recovery for the negative control (MRD control) was maintained over the testing period, with CFU/mL being over 4 orders of magnitude larger than for the hypochlorous acid samples for all concentrations and time points tested.

Overall, despite the extremely low hypochlorous acid concentrations used, all solutions showed rapid antimicrobial activity against the challenging organism E. coli NCIMB 14067.

### Example 9—Antimicrobial Efficacy of Sulfanilamide (Comparative Compound—Not of the Invention)

For comparison purposes with the media of the invention, the sterilisation potential of a known antimicrobial agent was investigated.

An in vitro direct inoculation method was used to assess the antimicrobial activity of sulfanilamide in an intermittent urinary catheter scenario against gram-positive Escherichia coli NCIMB 14067 and gram-negative Enterococcus faecalis NCTC 12201; two microorganisms associated with urinary tract infections. Sulfanilamide concentrations of 0.1% and 0.01% were tested and bacterial levels monitored over a period of 5 minutes.

Representative colonies of the challenge microorganisms, from an 18-24 hour challenge culture plate, were dispersed in MRD to obtain an optical density (OD₅₄₀ₙₘ) equivalent to approximately 1×10⁸ CFU/mL. This was diluted to obtain a working concentration of 1×10⁶ CFU/mL. A quantitative count was performed to confirm the inoculum level.

The challenge organism was prepared in MRD to a concentration of approximately 1×10⁶ CFU/mL. This suspension was added in a 1 mL volume to 9 mL of sample (n=3 per sample, n=2 per control) in a sterile 30 mL universal and pipetted up and down to mix. Following each testing period, 5 seconds, 30 seconds, 1 minute, 2 minutes and 5 minutes, 100 μL of the test solution was transferred to 0.9 mL and 9.9 mL volumes of DENB (1:10 and 1:100 dilutions respectively). The most appropriate dilutions were inoculated (100 μL) onto duplicate pre-dried (tryptone soy agar) TSA plates and the inoculum spread using separate sterile L-shaped spreaders (for 10⁻¹ and 10⁻² dilutions, 0.5 mL were placed onto duplicate plates). The TSA plates were allowed to dry before being inverted and incubated at 35±3° C. for at least 48 hours. Following the incubation period, the number of bacterial colonies were counted on the most appropriate dilution for each dressing type i.e. between 25-250 CFU/plate.

The samples tested were: 0.1% sulfanilamide in sterile deionised water (SDW); 0.01% sulfanilamide in SDW; 100 ppm hypochlorous acid in SDW; and a SDW control.

There was no decrease in challenge organism numbers of E. coli NCIMB 14067 or E. faecalis NCTC 12201 when added to sulfanilamide (at both concentrations tested) when compared to the control for any of the timepoints tested. In contrast, when testing, 100 ppm hypochlorous acid solution resulted in undetectable numbers of both challenge organisms within the shortest timepoint of 5 seconds and these undetectable numbers were then observed for the remaining testing period.

### CONCLUSIONS

Overall, the above tests have shown that the setup of the invention facilitates simple and safe catheter reuse.

The medium comprising chlorine-containing species allows for highly effective catheter lubrication and sterilisation to be achieved in a single step by as simply as contacting the catheter with the medium, such as by submerging the catheter in the medium. The medium also provides for long-term sterilisation and/or lubrication, allowing the catheter to be reused safely for relatively long periods of time, without the need for overly frequent re-sterilisation and/or re-lubrication and without the need to replace the medium frequently.

As such, the setup allows for a catheter to be removed from a resealable container for use and then sterilised/lubricated after use by simply reinserting the catheter back into the container and bringing the catheter into contact with the medium comprising chlorine-containing species in the container. The catheter can then be simply removed from the container again for the next use without the need for any further sterilisation/lubrication steps.

The present inventors have shown that this reuse/sterilisation cycle can be repeated multiple times with a single catheter. They have further shown that even without replacing the medium contained in the container for 24 hours, the catheter is still effectively sterilised and safe to use by merely contacting it with the medium.

The above embodiments are described by way of example only. Many variations are possible without departing from the scope of the invention as defined in the appended claims.

1. A reusable urinary catheter kit comprising a container comprising: a urinary catheter; and a medium comprising at least one chlorine-containing species, wherein the container is resealable.

2. A reusable urinary catheter kit as claimed in claim 1, wherein the catheter is an intermittent urinary catheter.

3. A reusable urinary catheter kit as claimed in any preceding claim, wherein the catheter further comprises at least one additive, preferably at least one amphiphilic additive, wherein the amphiphilic additive is preferably polymeric or oligomeric.

4. A reusable urinary catheter kit as claimed in claim 3, wherein the amphiphilic additive is an amphiphilic A-B block copolymer comprising a hydrophobic hydrocarbon A-block and a hydrophilic B-block.

5. A reusable urinary catheter kit as claimed in claim 4, wherein the amphiphilic additive is an A-B block copolymer comprising an A-block comprising a hydrocarbon chain block of the formula CH₃CH₂(CH₂CH₂)ₐ where “a” is 5-25 and preferably 9-25, and a hydrophilic B-block

6. A reusable urinary catheter kit as claimed in claim 4 or 5, wherein the B-block is a hydrophilic oligomer comprising between 2 and 10 monomer units optionally derived from monomers chosen from: alkylene oxides, alkylene glycols, epihalohydrins, unsaturated carboxylic acids, alkylene imines, lactones, vinyl alcohol, and vinyl alkanoates.

7. A reusable urinary catheter kit as claimed in any preceding claim, wherein the catheter comprises a hollow polymeric tubular body comprising a base polymer that is independently chosen from: polyolefins, polyesters, polyacrylates, polyamides, thermoplastic elastomeric material, polyether block amide, thermoplastic vulcanizates, thermoplastic copolyesters, thermoplastic polyamides, fluororubber, water disintegrable or enzymatically hydrolysable material, and combinations, blends or copolymers of any of the above materials, and wherein the base polymer preferably comprises a polymer independently chosen from: polyolefins, polyvinyl chloride, polyurethane, styrene-butadiene copolymer (SBC), styrene-ethylene-butylene-styrene copolymer (SEBS), thermoplastic elastomeric material, and combinations, blends or copolymers of any of the above materials.

8. A reusable urinary catheter kit as claimed in any preceding claim, wherein the medium comprises at least one chlorine-containing species that is independently chosen from: hypochlorous acid, at least one hypochlorite salt, chlorine dioxide, molecular chlorine, a dichloroisocyanurate salt, chloroazodin, dichlorodimethylhydantoin, chloroxylenol, chlorhexidine, and combinations thereof.

9. A reusable urinary catheter kit as claimed in claim 8, wherein the medium comprises at least one chlorine-containing species that is independently chosen from: hypochlorous acid, at least one hypochlorite salt, chlorine dioxide, and combinations thereof.

10. A reusable urinary catheter kit as claimed in claim 9, wherein the medium comprises one or more of: chlorine dioxide, hypochlorous acid, and at least one hypochlorite salt in a total combined concentration of between 1-100 ppm.

11. A reusable urinary catheter kit as claimed in claim 9 or 10, wherein the medium comprises at least one chlorine-containing species comprising hypochlorous acid and at least one further chlorine-containing species.

12. A reusable urinary catheter kit as claimed in claim 11, wherein the medium comprises hypochlorous acid and at least one hypochlorite salt.

13. A reusable urinary catheter kit as claimed in any one of claims 9 to 12, wherein the medium comprises at least one chlorine-containing species comprising hypochlorous acid and/or chlorine dioxide.

14. A reusable urinary catheter kit as claimed in any one of claims 9 to 13, wherein the medium comprises molecular chlorine and at least one of: hypochlorous acid and a hypochlorite salt.

15. A reusable urinary catheter kit as claimed in any preceding claim, wherein the medium comprises the at least one chlorine-containing species in a total concentration of between 0.005-0.6 wt. % of the medium.

16. A reusable urinary catheter kit as claimed in any preceding claim, wherein the medium comprises at least one alkali metal halide salt, preferably comprising sodium chloride, and preferably sodium chloride and at least one of: hypochlorous acid and a hypochlorite salt.

17. A reusable urinary catheter kit as claimed in claim 16, wherein the medium comprises at least one hypochlorite salt and further comprises at least one alkali metal halide salt, preferably comprising sodium chloride, wherein the ratio of the total concentration of the at least one alkali metal halide salt to the at least one hypochlorite salt in the medium is between 10-20.

18. A reusable urinary catheter kit as claimed in any preceding claim, wherein the medium comprises a base, preferably sodium hydroxide, and the medium preferably comprises at least one base, preferably comprising sodium hydroxide and at least one of: hypochlorous acid and a hypochlorite salt.

19. A reusable urinary catheter kit as claimed in any preceding claim, wherein the medium is present as a solution, preferably an aqueous solution.

20. A reusable urinary catheter kit as claimed in any preceding claim, wherein the medium is present as a liquid having a viscosity of between 0.5-5000 cP.

21. A reusable urinary catheter kit as claimed in any preceding claim, wherein the medium has a pH of between 2 and 14.

22. A reusable urinary catheter kit as claimed in any preceding claim, wherein the medium is in direct contact with the catheter, preferably with at least one surface thereof, and wherein the catheter is preferably submerged in the medium.

23. A reusable urinary catheter kit as claimed in any preceding claim, wherein the medium is contained in a separate container that is located in the container and is not in direct contact with the catheter, and wherein the separate container is preferably rupturable or pierceable, in use, to release the contained medium from the separate container and into direct contact with the catheter.

24. A method of sterilising and/or lubricating a reusable urinary catheter, the method comprising the steps of:
a. Providing a resealable container in its sealed configuration, said container comprising a urinary catheter, and further providing a medium comprising at least one chlorine-containing species;
b. Unsealing the container;
c. Removing the urinary catheter from the unsealed container;
d. Performing catheterisation with the catheter;
e. Reinserting the catheter into the container and contacting the catheter with the medium comprising at least one chlorine-containing species in the container; and
f. Resealing the container.

25. A method as claimed in claim 24, wherein the medium is contained in the container in step (a) of the method.

26. A method as claimed in claim 24, wherein the method further comprises the step of adding the medium into the container after step (b) and before reinserting the catheter into the container in step (e).

27. A method as claimed in claim 24, wherein the method further comprises the step of adding the medium into the container after reinserting the catheter into the container in step (e).

28. A method as claimed in any one of claims 24 to 27, wherein step (c) comprises contacting at least one surface of the catheter with the medium, preferably at least an outer surface of the catheter.

29. A method as claimed in any one of claims 24 to 28, wherein the method further comprises repeating steps (b) to (f) for each subsequent use of the catheter.
